(19) United States
(12) Patent Application Publication
(10) Pub. No.: US 2007/0037183 A1
(43) Pub. Date: Feb. 15, 2007

(54) Diagnostic and therapeutic target for macular degeneration

(75) Inventors: Albert O. Edwards, Dallas, TX (US); Lindsay A. Farrer, Ashland, MA (US)

Correspondence Address: Ronald I. Eisenstein;NIXON PEABODY LLP, 100 Summer Street, Boston, MA, 02110, US

(73) Assignees: Trustees of Boston University, Boston, MA (US); Board of Regents, The University of Texas System, Austin, TX (US)

(21) Appl. No.: 11/369,402

(22) Filed: Mar. 7, 2006

Related U.S. Application Data
(60) Provisional application No. 60/659,334, filed on Mar. 7, 2005.

(51) Int. Cl. C12Q 1/68

(52) U.S. Cl. 435/6

(57) ABSTRACT

The present invention is based on the discovery of genetic polymorphisms that are associated with ocular diseases and disorders, such as age-related macular degeneration (AMD). In particular, the present invention relates to methods for determining an individuals susceptibility to ocular disorders such as AMD by screening for mutations and/or polymorphisms in the human complement factor H (CFH) gene or gene product that confer susceptibility to such disorders. Also encompassed in the present invention are nucleic acid molecules containing the polymorphisms, variant proteins encoded by such nucleic acid molecules, reagents for detecting the polymorphic nucleic acid molecules and proteins, and methods of treatment following detection of susceptibility.

### CROSS REFERENCE TO RELATED APPLICATIONS

This application claims the benefit under 35 U.S.C. 119(e) of U.S. Provisional Patent Application Ser. No. 60/659,334, filed Mar. 7, 2005, the contents of which are herein incorporated by reference in their entirety.

### GOVERNMENT SUPPORT

This invention was made with Government Support under Contract Number EY014467, awarded by the National Institutes of Health. The Government has certain rights in the invention.

### FIELD OF THE INVENTION

The present invention is directed to methods for the diagnosis and determination of susceptibility for ocular disorders such as age-related macular degeneration, as well as methods for screening for novel therapies and methods for improved therapies for the treatment of age-related macular degeneration.

### BACKGROUND OF THE INVENTION

Age-related macular degeneration (AMD) is a leading cause of blindness in older individuals (1). It is a late-onset, complex trait with hereditary, lifestyle, and medical risk factors (2). The condition typically begins to be seen in the fifth decade of life with small yellow deposits external to the outer retina and retinal pigment epithelium (RPE) called drusen. Large numbers of drusen and clinical features of damage to the RPE markedly increase the risk of complications (atrophy of the RPE and abnormal neovascularization of the outer retina), leading to severe vision loss (1).

Although the primary pathogenic mechanisms of AMD remain unknown, there is strong evidence that genetics plays a role (3-9). The first locus for AMD (ARMD1) was reported in a single extended family linked to chromosome 1q25.3-31.3 (5).

While it has been proposed that a threshold event occurs during normal aging which leads to AMD, the sequelae of biochemical, cellular, and/or molecular events leading to the development of AMD are poorly understood. Drusen, pathological deposits that form between the retinal pigmented epithelium (RPE) and Bruch's membrane, are significant risk factors for the development of AMD. The characterization of the molecular composition of drusen has suggested that proteins associated with inflammation and immune-mediated processes are prevalent among drusen-associated constituents. For example, proteins and transcripts that encode a number of these molecules have been detected in retinal, RPE, and choroidal cells. Hageman et al., Prog Retin Eye Res. 2001 Nov; 20(6):705-32.

More specifically, it has been suggested that combined events of cellular dysfunction and death, drusen deposition and activation of immunomodulatory processes at the level of the RPE-choroid-retina complex are key elements that participate in AMD. Cellular remnants and debris derived from degenerate RPE cells become sequestered between the RPE basal lamina and Bruch's membrane. This cellular debris has been suggested to constitute a chronic inflammatory stimulus, and a potential “nucleation” site for drusen formation. The entrapped cellular debris then becomes the target of encapsulation by a variety of inflammatory mediators, some of which are contributed by the RPE and, perhaps, other local cell types; and some of which are extravasated from the choroidal circulation. One set of immunomodulartory molecules which have been proposed to play some role in the development of AMD are the proteins of the complement system. See for example U.S. published patent application numbers 20030017501, 20020102581, 20030149997, 20020015957, and 20030207309, and WO 03071927.

The complement system consists of a group of globulins in the serum of humans (Hood, L. E. et al. 1984, Immunology, 2d Edition, The Benjamin/Cummings Publishing Co., Menlo Park, Calif., p. 339; See also, U.S. Pat. Nos. 6,087,120 and 5,808,109). Complement activation plays an important role in the mediation of immune and allergic reactions (Rapp, H. J. and Borsos, T., 1970, Molecular Basis of Complement Action, Appleton-Century-Crofts (Meredith), N.Y.). The activation of complement components leads to the generation of a group of factors, including chemotactic peptides that mediate the inflammation associated with complement-dependent diseases. The activities mediated by activated complement proteins include lysis of target cells, chemotaxis, opsonization, stimulation of vascular and other smooth muscle cells, degranulation of mast cells, increased permeability of small blood vessels, directed migration of leukocytes, and activation of B lymphocytes, macrophages and neutrophils (Eisen, H. N., 1974, Immunology, Harper & Row, Publishers, Inc., Hagerstown, Md., p. 512).

The “alternative pathway” of the complement system provides natural, non-immune defense against microbial infections. In addition, this pathway amplifies antibody-antigen reactions. Alternative pathway recognition occurs in the presence of C3b and an activating substance such as bacterial lipoprotein, surfaces of certain parasites, yeasts, viruses and other foreign body surfaces, such as biomaterials. The alternative pathway of the complement system involves the recognition of certain polysaccharides (e.g., on microbial surface) and is activated by the presence of a specific substrate called C3bB, a complex of complement proteins. See, e.g., Cooper, Adv Immunol, 37(-HD-):151-216, 1985; Fearon & Austen, J. Exp. Med. 146: 22-33, 1977; Pangburn et al., 266: 16847-53, 1991; Matsushita et al., Microbiol Immunol, 40(12):887-93, 1996; and Turner et al., Res Immunol, 147(2): 110-5, 1996. The main alternative pathway components are designated factor B, factor D, Properdin, factor H and factor I.

In recent years, a significant number of mutations have been identified in the gene encoding human Factor H, known as HF1 (Rodriguez Cordoba et al., Molec. Immunol. 41: 355-367 (2004)), which has revealed an association with two different renal diseases, glomerulonephritis and atypical hemolytic uremic syndrome (aHUS). Factor H deficiencies in mouse and humans have been described, including mutations that result in truncations or amino acid substitutions that impair secretion of factor H into the circulation (Ault et al., J. Biol. Chem. 272: 25168-75 (1997); Sanchez-Corral et al., Immunogenetics 51:366-399 (2000); Hegasy et al., Immunogentics 55:462-71 (2003). Lack of factor H in plasma causes uncontrolled activation of the alternative pathway, with consumption of C3 and often other terminal complement components (Thompson et al., Clin. Exp. Immunol. 46: 110-119 (1981); Ault et al. 1997).

No gene(s) has yet been identified that causes a significant proportion of AMD. Moreover, no major molecular pathways involved in the etiology of this disease have been elucidated. It would be desirable to identify such genes and methods of determining which individuals are at increased risk for developing AMD.

### SUMMARY OF THE INVENTION

The present invention provides novel methods for screening individuals for increased susceptibility to, or current affliction with, a disease or disorder associated with a variance (e.g., mutation or polymorphism) in the gene encoding complement factor H (CFH). Such complement factor H-associated diseases or disorders include eye diseases and disorders, including age-related macular degeneration (AMD), optic nerve disorders and cardiovascular disease. Preferably, the disease or disorder is AMD.

In one embodiment, the methods comprise obtaining a biological sample from an individual and screening for variations (e.g. changes) in the human CFH gene or gene product relative to a control group (e.g. wildtype, positive and/or negative control group). The presence of a variance (e.g. a change, mutation, polymorphism, or SNP) in the human CFH gene or CFH protein in the biological sample, as compared to the control group, indicates susceptibility to, or current affliction with, a CFH-associated disease or disorder. In a preferred embodiment the CFH-associated disease or disorder is AMD.

We have discovered that polymorphisms in the human gene encoding complement factor H, CFH, are highly associated with AMD. For example, possession of at least one histidine (a change from tyrosine) at amino acid position 402 of SEQ ID NO: 2 increases the risk of AMD 2.7-fold and may account for 50% of the attributable risk of AMD. Thus, in one embodiment, the AMD-predisposing allele is a polymorphism or mutation within the coding portion of the CFH gene. In one preferred embodiment, the AMD-predisposing allele is a polymorphism or mutation which encodes an amino residue located within a short consensus repeat (SCR) of CFH; one preferred SCR is SCR7. In one particularly preferred embodiment, the AMD-predisposing allele of the CFH gene is a change from tyrosine at amino acid 402 of CFH (SEQ ID NO:2). Even more preferably, the polymorphism changes a tyrosine at position 402 to a histidine.

The presence or absence of the polymorphisms (also known as AMD-predisposing alleles) can be determined by any means known in the art. Preferably, one screens the CFH gene for any changes in the nucleic acid sequence. Preferably, the nucleotide to be screened is at position 1277 of SEQ ID NO: 1. A change at this position from a thymine to a cytosine indicates susceptibility to, or current affliction with, AMD. Alternatively, one can screen complement factor H protein for any changes in the amino acid sequence. Here, a change at amino acid number 402 of SEQ ID NO: 2 (e.g. from a Tyrosine to a Histidine) is indicative of susceptibility to, or current affliction with, AMD.

In one embodiment, a probe is used to screen for variances (e.g., mutations and/or polymorphisms) in the human CFH gene. Variances in the human CFH gene may also be determined via sequence analysis, such as, for example, amplification assays such PCR, qPCR, rtPCR, or gene arrays. Alternatively, variances in the human CFH gene may also be detected in the CFH gene product (e.g. mRNA or protein). Probes may be useful to screen for variances in the human CFH protein.

In one embodiment of the present invention, a biological sample from a patient with or at risk for developing ocular or cardiovascular disease and/or disorders is analyzed. A variance in the human CFH gene is indicative of the presence of or the possibility of future affliction with, a complement factor H-associated diseases or disorders including eye diseases and disorders, including age-related macular degeneration (AMD), optic nerve disorders and cardiovascular disease. Preferably, the variance a change from tyrosine at amino acid 402 of CFH to a histidine.

The detection of the presence or absence of at least one nucleic acid variance can be determined by amplifying a segment of nucleic acid encoding human CFH. The segment to be amplified is 1000 nucleotides in length, preferably, 500 nucleotides in length, and most preferably 100 nucleotides in length or less. The segment to be amplified can include a plurality of variances.

In another embodiment, the presence or absence of a variance in the human CFH gene can be detected by analyzing the gene product (e.g., protein). In one embodiment, a probe that specifically binds to a variant CFH is utilized. In a preferred embodiment, the probe is an antibody that preferentially binds to a variant CFH. The presence of a variant CFH predicts the likelihood of susceptibility to a complement factor H-associated diseases or disorder. Alternatively, the probe may be an antibody fragment, chimeric antibody, humanized antibody or an aptamer.

The present invention further provides a novel method for treating a patient affected with or at risk for developing an ocular disease or disorder, such as, for example, AMD. The method involves determining whether the human CFH gene of a patient contains at least one nucleic acid variance. Preferably, the variance is a change from tyrosine at amino acid 402 of CFH to a histidine. The presence of such a variance indicates that a CFH targeted treatment will be effective. If the variance is present, a suitable treatment plan may be initiated.

Suitable treatments plans are known to those of skill in the art and include, for example, the administration of angiogenesis inhibitors, VEGF inhibitors and the like. Preferably, a VEGF inhibitor is administered to a patient identified with a variance in CFH gene or gene product. Preferably, the VEGF inhibitor is ranibizumab (Lucentis™), a humanized antibody fragment designed to bind to and inhibit the activity of VEGF. In another embodiment, after diagnosis of a mutation or polymorphism in the human CFH gene, a photodynamic laser therapy (e.g. Visudyne®) and/or pegaptanib sodium (Macugen®) treatment is initiated. Other suitable treatment plans are known to those of skill in the art and are encompassed herein. In a preferred embodiment, treatment is initiated before the onset of symptoms (e.g. immediately after diagnosis of a mutation and/or polymorphism in CFH gene) and thus the dosages used in the treatment may be reduced.

### BRIEF DESCRIPTION OF THE DRAWINGS

FIG. 1 shows that the regulation of complement activation (RCA) locus located within chromosome 1q31.3 includes the gene for complement factor H (CFH), 5 related genes derived from CFH through ancestral duplications, and the gene for factor 13B (F13B). A megabase (Mb) scale of this region is provided at the top of the figure. SNPs genotyped across the RCA locus are shown along the bottom of the figure. The negative natural logarithm of the significance of allele association to AMD for each SNP is given in the graph (10). The 0.05 significance level is shown by the dotted line. Values greater than 15 on the y-axis correspond to P-values less than 10⁻⁷.

FIG. 2 shows pairwise linkage disequilibrium (LD) among 38 SNPs in and surrounding the RCA locus, spanning the region including the 5′ upstream region of CFH (represented by the SNP rs3753394) and the proximal portion of FRBZI (represented by the SNP rs10732295), a distance of 516 kb. LD was measured by the D′ statistic using data from all subjects in the discovery sample (10). A D′ value of 1 indicates complete linkage disequilibrium between two markers and a D′ value of 0 indicates complete linkage equilibrium. Using the Haploview version 3.0 software, we divided the RCA locus into four haplotype blocks (29). D′ values for all, or nearly all, pairwise comparisons within a haplotype block are at least 0.8 (black squares). The haplotype block structure is similar to that obtained by the HapMap Project for this region in Caucasians with the notable difference that blocks 2 and 3 in the our structure comprise one block in the HapMap report (10). The lone SNP (rs379370) between blocks 2 and 3 is a nscSNP in the CFHL4 gene (Table 3) showing moderate LD (gray squares), modest LD (light gray squares), or low LD (white squares) with the SNPs in block 3.

FIG. 3 shows that the ARMD1 locus spans 14 megabases (Mb) based on meiotic breakpoint mapping (Table 3, 19) and includes 46 genes arranged in 6 clusters. Short tandem repeat markers delineating and confirming the ARMD1 locus in linkage studies are provided underneath the Mb scale. The number of SNPs genotyped is provided under each cluster. Three simultaneous strategies were pursued for genotyping across the ARMD1 locus: common nscSNPs, exclusion of the Fibulin-6 candidate gene (S19), and gene-based SNPs at 8 kb to 25 kb density. After association of AMD to the regulation of complement activation locus (genes CFH through F13B in cluster 6) was identified, new SNP assays were not designed for other regions of the genome. For this reason, no gene-based genotyping was performed in clusters 2 and 5.236 SNPs are reported herein and in Table 4.

FIG. 4 shows the nucleic acid sequence of the gene, CFH, encoding human complement factor H (SEQ ID NO:1).

FIG. 5 shows the amino acid sequence of human complement factor H (SEQ ID NO:2).

FIG. 6 shows the alignment of SCR7 regions in complement factor H from four species, including human.

FIG. 7 shows the nucleic acid sequence of the gene, CFH, encoding human complement factor H (SEQ ID NO:9; Genbank Accession No. NM000186). By historical convention, the Genbank sequence is listed with a C at position 1277 that codes for the histidine variant the present invention associates with AMD. FIG. 6 shows conservation of Tyr at position 402 in the amino acid sequence. The histidine variant is the minor allele in the population.

FIG. 8 shows the amino acid sequence of human complement factor H (SEQ ID NO:10; Genbank Accession No. NP000177). As described above, by historical convention the Genbank sequence is listed with a Histidine at position 402. FIG. 6 shows conservation of Tyr at position 402. This histidine variant is the minor allele in the population.

### DETAILED DESCRIPTION OF THE INVENTION

We have discovered that variances (e.g. changes such as mutations and/or polymorphisms) in the human gene encoding complement factor H, CFH, from the wildtype sequence are highly associated with age-related macular degeneration (AMD). Accordingly, the present invention provides novel methods for screening for individuals with increased susceptibility to, or current affliction with, a disease or disorder associated with a mutation or polymorphism in the gene encoding complement factor H in comparison to the population at large. Complement factor H-associated diseases include eye diseases, including age-related macular degeneration (AMD), optic nerve disorders and cardiovascular disease. For example, using two independent case-control populations, significant association (P=4.95×10⁻¹⁰) with AMD was identified within the regulation of complement activation locus, and centered over a Tyr402His protein polymorphism in the gene encoding complement factor H. As described in detail below, possession of at least one allele encoding histidine at amino acid 402 increased the risk of AMD 2.7-fold, and accounts for 50% of the attributable risk of AMD.

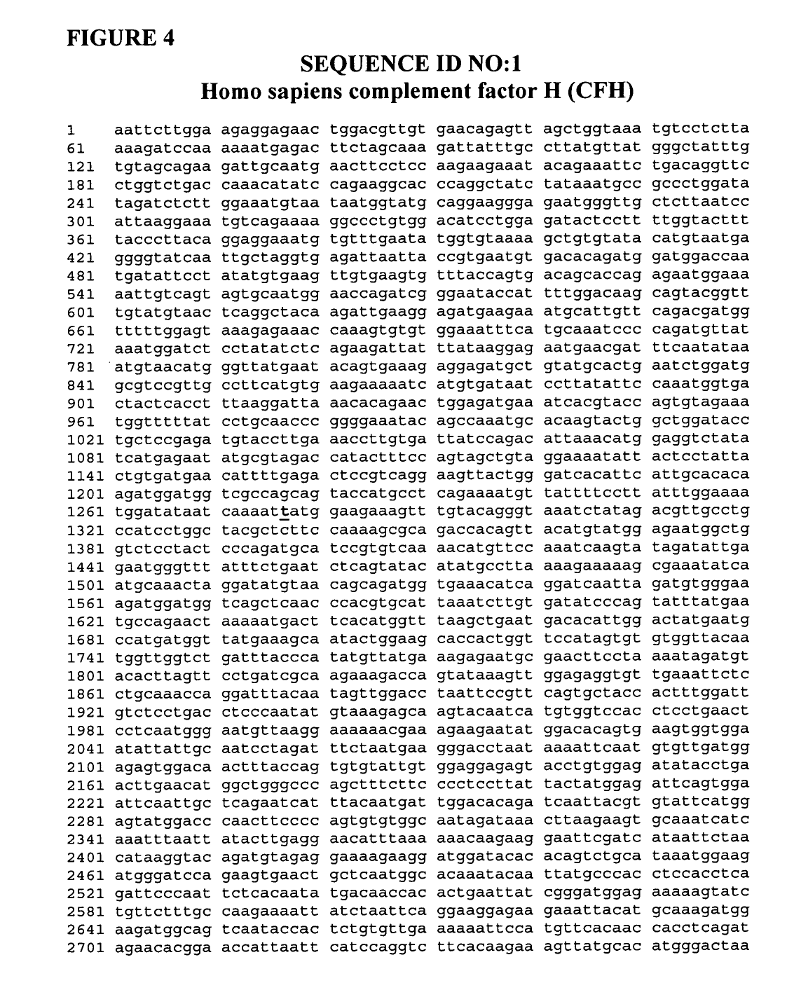

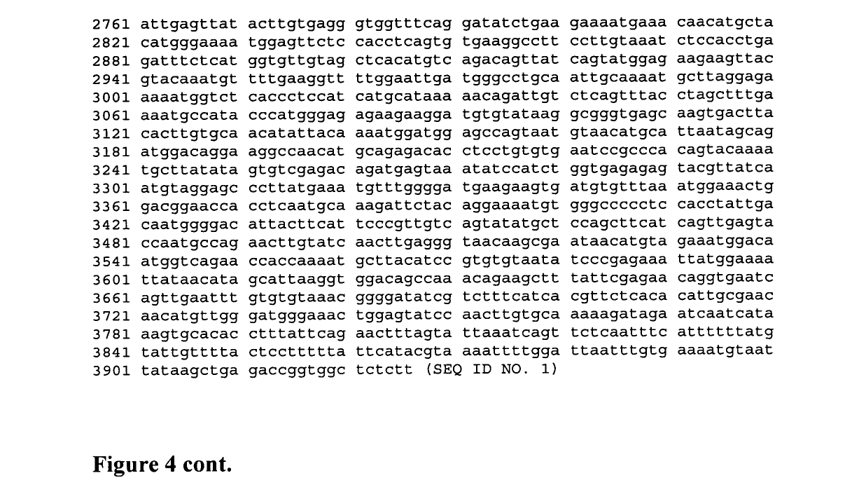

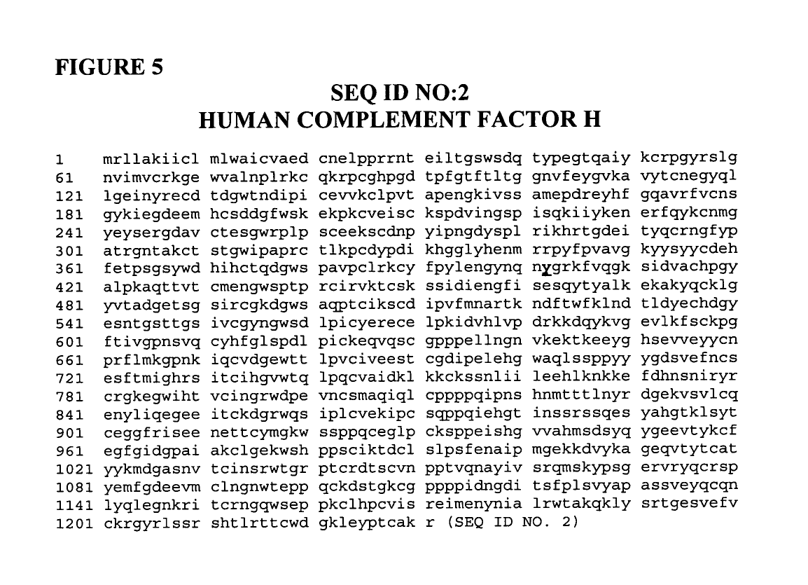

A single nucleotide polymorphism (SNP) in the gene encoding human complement factor H, in which a cytosine is present at position 1277 in SEQ ID NO: 1 (as opposed to the wildtype (e.g. negative control) shown in FIG. 4, where a thymine is present at position 1277), that codes for a histidine instead of a tyrosine at amino acid 402 of CFH is responsible for increased susceptibility to AMD. The presence of such C-allele in the CFH gene in an individual is predictive of increased susceptibility to AMD, whereas a T allele is protective. This polymorphism is sometimes referred to as “T1277C” or “Y402H”. A single histidine allele (heterozygous) confers an increased risk of AMD, whereas two histidine alleles (homozygous) confers an even greater predictability of risk. The mutation is in a region of CFH that binds to both heparin and C-reactive protein. Furthermore, the presence of such a mutation may confirm the presence of AMD, for example at early stages of disease when symptoms may not be evident.

The screening methods of the present invention predict susceptibility to atrophic, exudative, geographic and neovascular AMD. In a preferred embodiment, the presence of the T1277C polymorphism is predictive of future onset of exudative AMD. Furthermore, the methods of the present invention may be combined with other diagnostic methods known to those of skill in the art or those to be discovered subsequently.

The present invention also provides novel methods of screening individuals to determine if they have an increased susceptibility to cardiovascular disorders in comparison to the population at large, by screening for mutations and/or polymorphisms in the gene encoding human complement factor H. Risk factors shared by cardiovascular disease and AMD include a high fat diet, lack of exercise, diet, elevated CRP levels and obesity. In addition, there is increased cardiovascular mortality in patients with advanced AMD. See AREDS Report No. 13, Arch. Ophthalmol. 122: 716-726 (2004). The presence of the T1277C polymorphism indicates increased susceptibility to cardiovascular disease, including, but not limited to, myocardial infarction, ischaemic stroke, and venous thromboembolism.

In one embodiment, the patient population screened is Caucasian. In a preferred embodiment, the patient population is Caucasian and at least age 45, more preferably age 50, even more preferably age 55, 60, 65, 70, etc. Any race and age may be screened and individuals may be chosen by their physician according to the presence of risk factors such as, for example, age, ethnicity, smoking habits, hypertension, obesity, familial history of AMD and diet. Other risk factors are known to those of skill in the art.

In one embodiment, the present methods involve using a probe to screen for variances (e.g. changes, mutations, polymorphisms, SNPs) in the human CFH gene or gene products, and its variants from alternative splicing or other means, relative to a control group (e.g. wildtype sequence).

According to the present invention, a “baseline” or “control” or “control group” can include a normal or negative control and/or a disease or positive control, against which a test sample can be compared. Therefore, it can be determined, based on the control, whether a sample to be evaluated for mutations and/or polymorphisms in the human CFH gene has a measurable difference or substantially no difference, as compared to the control group. In one aspect, the baseline control is a negative control. The negative control has a CFH gene as expected in the sample of a normal (e.g., healthy, negative control) individual. Therefore, the term “negative control” used herein typically refers to a population of individuals whose CFH sequence is similar to SEQ ID NO. 1, namely, a t at position 1277, which is believed to be normal (i.e., not having or developing susceptibility to ocular diseases or disorders). In some embodiments of the invention, it may also be useful to compare the gene expression in a test sample to a baseline that has previously been established from a patient or population of patients having susceptibility to ocular disorders. Such a baseline level, also referred to herein as a “positive control”, refers to a CFH gene expression established from one or preferably a population of individuals who has been positively diagnosed with or having susceptibility to ocular disorders and whom have CFH sequence similar to SEQ ID NO: 9 (e.g. a c instead of a t at position 1277).

Mutations and/or Polymorphisms in CFH

The present invention provides methods for determining changes at specific locations in DNA and/or protein sequence, typically referred to as polymorphisms or sometimes mutations, sometimes referred to as SNPs or polymorphic alleles, associated with AMD, nucleic acid molecules containing polymorphisms, methods and reagents for the detection of the changes in the wildtype sequence of CFH, uses of these polymorphisms for the development of detection reagents, and assays or kits that utilize such reagents. The AMD-associated polymorphisms disclosed herein are useful for diagnosing, screening for, and evaluating predisposition and prognosis to AMD and related pathologies in humans. The AMD-associated polymorphisms are also useful in detecting disease or disorder that is already present. A treatment regime can then be implemented. For example, administration of an anti-angiogenic agent. Preferably, one begins treatment as soon as possible. This is particularly important in early stages when it may be difficult to diagnose disease or disorder. Furthermore, such CFH proteins containing mutations or variations are useful targets for the development of therapeutic agents.

CFH encodes complement factor H, a single polypeptide chain plasma glycoprotein of 155 kDa. The secreted form of the protein is composed of 20 repetitive units of 60 amino acids, named short consensus repeats (SCR) arranged in a continuous fashion like a string of 20 beads (Ripoche al al., Biochem. J. 249: 593-602 (1988)). The SCRs have a typical framework of highly conserved residues including four cysteines, two prolines, one tryptophan, and several other partially conserved glycines and hydrophoblic residues. See Rodriguez de Cordoba et al., Molec. Immuno. 41:355-67 (2004). The Tyr402His polymorphism is located within SCR7, which contains the overlapping binding sites for heparin, C-reactive protein, and M-protein.

Re-interpretation of existing literature strongly suggests that the histidine variant of CFH is inactive in biologically important ways compared to the tyrosine variant of CFH. This prior work is consistent with reduced biological activity for the histidine variant of CFH. Two populations of CFH referred to as φ₁ and φ₂ can be isolated based on hydrophobic affinity chromatography (J. Ripoche, A. Al Salihi, J. Rousseaux, M. Fontaine, Biochem J 221, 89 (Jul. 1, 1984)). Both populations have identical polypeptide chain length and interact equally well with C3b, but φ₂ is bound by phenyl-Sepharose while φ₁ is slightly retarded on the column (Riphoce et al., 1984). Only the φ₂ population induced aggregation of sheep red blood cells coated with complement components, bound specifically to a human B lymphoblastoid cell line, and induced secretion of lymphocyte-activating factor by human monocytes leading to the suggestion that φ₂ might represent an activated form of CFH (Ripoche et al., 1984; J. Ripoche et al., Biochem J 253, 475 (Jul. 15, 1988).; D. Iferroudjene, M. T. Schouft, C. Lemercier, D. Gilbert, M. Fontaine, Eur J Immunol 21, 967 (April 1991). Additional experiments showed differing immunoreactivity to monoclonal antibodies and conversion of the chromatographic properties of φ₂ to those of φ₁ by iodination of tyrosine residues, demonstrating that a change at one or more tyrosine residues accounted for the difference between φ₁ and φ₂ (Ripoche et al. 1988; C. Lemercier, O. Duval, J. Ripoche, R. B. Sim, M. Fontaine, Complement Inflamm 8, 181 (1991)). Most individual sera had equal proportions of φ₁ and φ₂, but some individuals had only one population consistent with a co-dominant protein polymorphism (Lemercier et al.). These observations suggest that φ₁ represents the histidine variant and φ₂ the tyrosine variant. We found that the replacement of tyrosine with histidine at amino acid 402 of CFH, results in decreased inhibition of the alternative complement pathway in the outer retina and choroid. A lifetime of abnormal regulation of inflammation gives rise to the formation of subretinal deposits and eventually AMD. This hypothesis is further supported by the observation of drusen with terminal complement deposition indistinguishable from AMD in eyes from patients with a kidney disease (membranoproliferative glomerulonephritis type II) that can be caused by mutations in CFH (R. F. Mullins, N. Aptsiauri, G. S. Hageman, Eye 15, 390 (June 2001); M. A. Dragon-Durey et al., J Am Soc Nephrol 15, 787 (March 2004)).

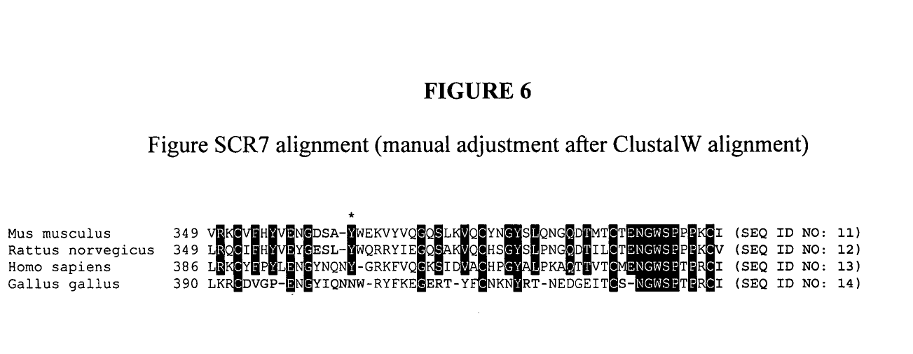

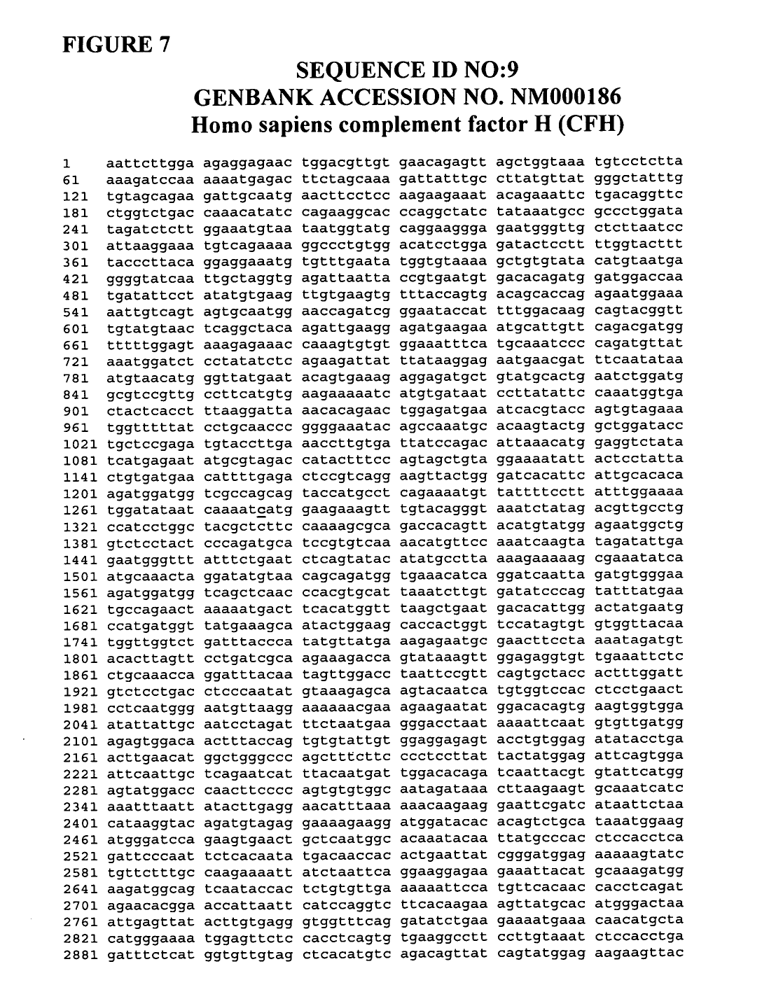

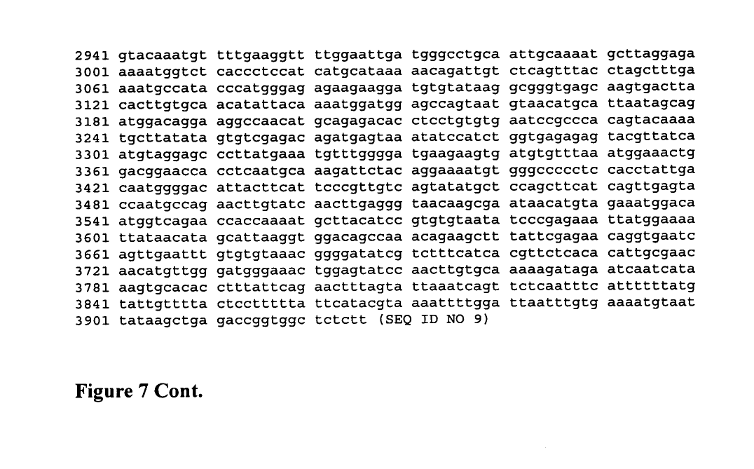

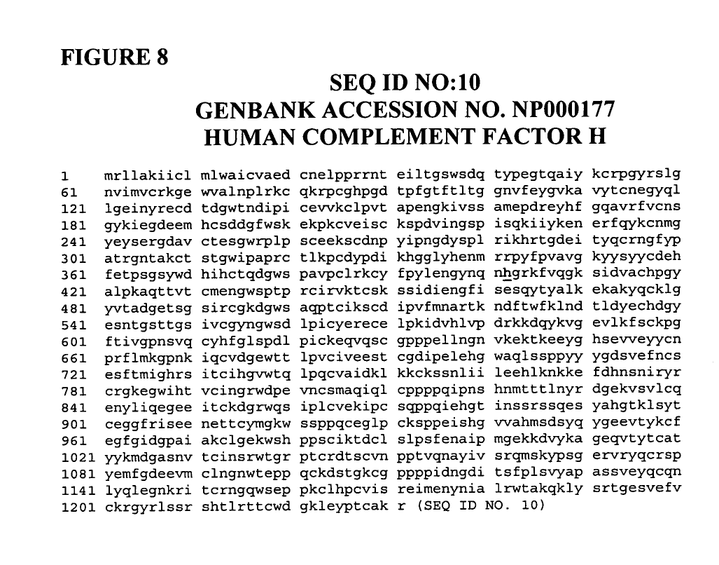

In one embodiment, the AMD-predisposing allele is a polymorphism or mutation within the coding portion of the CFH gene (SEQ ID NO:1). In one preferred embodiment, the AMD-predisposing allele is a polymorphism or mutation which encodes an amino acid residue located within a short consensus repeat (SCR) of CFH; one preferred SCR is SCR7, as shown in FIG. 6. In one particularly preferred embodiment, the AMD-predisposing allele of the CFH gene is a change from tyrosine at amino acid 402 of CFH. Even more preferably, the polymorphism changes a tyrosine at position 402 to a histidine.

In one embodiment, one can also look for such changes in the corresponding complement factor H gene product (SEQ ID NO:2). This can be readily done by standard means such as antibodies that recognize specific epitopies. In one embodiment one can use an antibody to the C-terminus to recognize mutations resulting in truncations of the protein or changes in its level of expression. One can also generate antibodies that will only recognize specific amino acid sequences. For example, antibodies that recognize the polymorphic protein (or protein fragment) of SEQ ID NO: 10 and does not recognize the wildtype protein (or protein fragment) of SEQ ID NO: 2 are encompassed. Antibodies and antibody fragments, polyclonal or monoclonal, can be purchased from a variety of commercial suppliers, or may be manufactured using well-known methods, e. g., as described in Harlow et al., Antibodies: A Laboratory Manual, 2nd Ed; Cold Spring Harbor Laboratory Press, Cold Spring Harbor, N.Y. (1988).

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest. Preferably, the antibody is diagnostic in that it discriminatively binds to either the wildtype or the AMD-predisposing allele of CFH described herein.

An AMD-predisposing allele maybe located within a coding region of CFH or a non-coding region of CFH. Non-coding regions include, for example, intron sequences as well as 5′ and 3′ untranslated sequences. In one preferred embodiment, the AMD-predisposing allele is located within a portion of the CFH gene which encodes SCR7. Changes of interest in a non-coding region include modifications of the nucleic acid such as methylation.

Another embodiment of the invention provides methods for identifying novel polymorphisms in the human CFH gene which are associated with AMD. The strength of the association between a polymorphic allele and AMD can be characterized by a particular odds ratio such as an odds ratio of at least 2 with a lower 95% confidence interval limit of greater than 1. Such an odds ratio can be, for example, at least 3.0, 4.0, 5.0, 6.0, 7.0, or 8.0 or greater with a lower 95% confidence interval limit of greater than 1. In one embodiment, the predisposing polymorphic allele is associated with AMD with an odds ratio of at least 2 and a lower 95% confidence limit greater than 1. Methods for determining an odds ratio are well known in the art (see, for example, Schlesselman et al., Case Control Studies: Design, Conduct and Analysis Oxford University Press, New York (1982)).

In one embodiment, an AMD-predisposing allele is associated with the AMD with a p value of equal to or less than 0.05. In other embodiments, an AMD-predisposing allele is associated with AMD with a p value of equal to or less than 0.01. As used herein, the term “p value” is synonymous with “probability value.” As is well known in the art, the expected p value for the association between a random allele and disease is 1.00. A p value of less than about 0.05 indicates that the allele and disease do not appear together by chance but are influenced by positive factors. Generally, the statistical threshold for significance of linkage has been set at a level of allele sharing for which false positives would occur once in twenty genome scans (p=0.05). In particular embodiments, a AMD-predisposing allele is associated with AMD with a p value of equal to or less than 0.1, 0.05, 0.04, 0.03, 0.02, 0.01, 0.009, 0.008, 0.007, 0.006, 0.005, 0.004, 0.003, 0.002 or 0.001, or with a p value of less than 0.00095, 0.0009, 0.00085, 0.0008 or 0.0005. It is recognized that, in some cases, p values may need to be corrected, for example, to account for factors such as sample size (number of families), genetic heterogeneity, clinical heterogeneity, or analytical approach (parametric or nonparametric method).

Detection of Mutations or Polymorphisms in CFH

According to the present invention, any approach that detects mutations or polymorphisms in a gene can be used, including but not limited to single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes of the present invention (Michalatos-Beloin et al. (1996) Nucleic Acids Res. 24:4841-4843; Barnes (1994) Proc. Natl. Acad. Sci. USA 91:5695-5699: Ruano and Kidd (1991) Nucleic Acids Res. 19:6877-6882).

In one embodiment, a long-range PCR (LR-PCR) is used to detect mutations or polymorphisms of the present invention. LR-PCR products are genotyped for mutations or polymorphisms using any genotyping methods known to one skilled in the art, and haplotypes inferred using mathematical approaches (e.g., Clark's algorithm (Clark (1990) Mol. Biol. Evol. 7:111-122).

For example, methods including complementary DNA (cDNA) arrays (Shalon et al., Genome Research 6(7):639-45, 1996; Bernard et al., Nucleic Acids Research 24(8):1435-42, 1996), solid-phase mini-sequencing technique (U.S. Pat. No. 6,013,431, Suomalainen et al. Mol. Biotechnol. Jun; 15(2): 123-31, 2000), ion-pair high-performance liquid chromatography (Doris et al. J. Chromatogr. A May 8;806(1):47-60, 1998), and 5′ nuclease assay or real-time RT-PCR (Holland et al. Proc Natl Acad Sci USA 88: 7276-7280, 1991), or primer extension methods described in the U.S. Pat. No. 6,355,433, can be used.

In one embodiment, the primer extension reaction and analysis is performed using PYROSEQUENCING™ (Uppsala, Sweden) which essentially is sequencing by synthesis. A sequencing primer, designed directly next to the nucleic acid differing between the disease-causing mutation and the normal allele or the different SNP alleles is first hybridized to a single stranded, PCR amplified DNA template from the individual, and incubated with the enzymes, DNA polymerase, ATP sulfurylase, luciferase and apyrase, and the substrates, adenosine 5′ phosphosulfate (AP S) and luciferin. One of four deoxynucleotide triphosphates (dNTP), for example, corresponding to the nucleotide present in the mutation or polymorphism, is then added to the reaction. DNA polymerase catalyzes the incorporation of the dNTP into the standard DNA strand. Each incorporation event is accompanied by release of pyrophosphate (PPi) in a quantity equimolar to the amount of incorporated nucleotide. Consequently, ATP sulfurylase converts PPi to ATP in the presence of adenosine 5′ pbosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAM™. Each light signal is proportional to the number of nucleotides incorporated and allows a clear determination of the presence or absence of, for example, the mutation or polymorphism. Thereafter, apyrase, a nucleotide degrading enzyme, continuously degrades unincorporated dNTPs and excess ATP. When degradation is complete, another dNTP is added which corresponds to the dNTP present in for example the selected SNP. Addition of dNTPs is performed one at a time. Deoxyadenosine alfa-thio triphosphate (dATPαS) is used as a substitute for the natural deoxyadenosine triphosphate (dATP) since it is efficiently used by the DNA polymerase, but not recognized by the luciferase. For detailed information about reaction conditions for the PYROSEQUENCING, see, e.g. U.S. Pat. No. 6,210,891, which is herein incorporated by reference in its entirety.

Another example of the methods useful for detecting mutations or polymorphisms is real time PCR. All real-time PCR systems rely upon the detection and quantification of a fluorescent reporter, the signal of which increases in direct proportion to the amount of PCR product in a reaction. Examples of real-time PCR method useful according to the present invention include, TaqMan® and molecular beacons, both of which are hybridization probes relying on fluorescence resonance energy transfer (FRET) for quantitation. TaqMan Probes are oligonucleotides that contain a fluorescent dye, typically on the 5′ base, and a quenching dye, typically located on the 3′ base. When irradiated, the excited fluorescent dye transfers energy to the nearby quenching dye molecule rather than fluorescing, resulting in a nonfluorescent substrate. TaqMan probes are designed to hybridize to an internal region of a PCR product (ABI 7700 (TaqMan™), Applied BioSystems, Foster City, Calif.). Accordingly, two different primers, one hybridizing to the mutation or polymorphism and the other to the corresponding wildtype allele, are designed. The primers are consequently allowed to hybridize to the corresponding nucleic acids in the real time PCR reaction. During PCR, when the polymerase replicates a template on which a TaqMan probe is bound, the 5′ exonuclease activity of the polymerase cleaves the probe. Consequently, this separates the fluorescent and quenching dyes and FRET no longer occurs. Fluorescence increases in each cycle, proportional to the rate of probe cleavage.

Molecular beacons also contain fluorescent and quenching dyes, but FRET only occurs when the quenching dye is directly adjacent to the fluorescent dye. Molecular beacons are designed to adopt a hairpin structure while free in solution, bringing the fluorescent dye and quencher in close proximity. Therefore, for example, two different molecular beacons are designed, one recognizing the mutation or polymorphism and the other the corresponding wildtype allele. When the molecular beacons hybridize to the nucleic acids, the fluorescent dye and quencher are separated, FRET does not occur, and the fluorescent dye emits light upon irradiation. Unlike TaqMan probes, molecular beacons are designed to remain intact during the amplification reaction, and must rebind to target in every cycle for signal measurement. TaqMan probes and molecular beacons allow multiple DNA species to be measured in the same sample (multiplex PCR), since fluorescent dyes with different emission spectra may be attached to the different probes, e.g. different dyes are used in making the probes for different disease-causing and SNP alleles. Multiplex PCR also allows internal controls to be co-amplified and permits allele discrimination in single-tube assays. (Ambion Inc, Austin, Tex., TechNotes 8(1)-February 2001, Real-time PCR goes prime time).

Yet another method useful according to the present invention for detecting a mutation or polymorphism is solid-phase mini-sequencing (Hultman, et al., 1988, Nucl. Acid. Res., 17, 4937-4946; Syvanen et al., 1990, Genomics, 8, 684-692). In the original reports, the incorporation of a radiolabeled nucleotide was measured and used for analysis of the three-allelic polymorphism of the human apolipoprotein E gene. The method of detection of the variable nucleotide(s) is based on primer extension and incorporation of detectable nucleoside triphosphates in the detection step. By selecting the detection step primers from the region immediately adjacent to the variable nucleotide, this variation can be detected after incorporation of as few as one nucleoside triphosphate. Labelled nucleoside triphosphates matching the variable nucleotide are added and the incorporation of a label into the detection step primer is measured. The detection step primer is annealed to the copies of the target nucleic acid and a solution containing one or more nucleoside triphosphates including at least one labeled or modified nucleoside triphosphate, is added together with a polymerizing agent in conditions favoring primer extension. Either labeled deoxyribonucleoside triphosphates (dNTPs) or chain terminating dideoxyribonucleoside triphosphates (ddNTPs) can be used. The solid-phase mini-sequencing method is described in detail, for example, in the U.S. Pat. No. 6,013,431 and in Wartiovaara and Syvanen, Quantitative analysis of human DNA sequences by PCR and solid-phase minisequencing. Mol Biotechnol 2000 June; 15(2):123-131.

Another method to detect mutations or polymorphisms is by using fluorescence tagged dNTP/ddNTPs. In addition to use of the fluorescent label in the solid phase mini-sequencing method, a standard nucleic acid sequencing gel can be used to detect the fluorescent label incorporated into the PCR amplification product. A sequencing primer is designed to anneal next to the base differentiating the disease-causing and normal allele or the selected SNP alleles. A primer extension reaction is performed using chain terminating dideoxyribonucleoside triphosphates (ddNTPs) labeled with a fluorescent dye, one label attached to the ddNTP to be added to the standard nucleic acid and another to the ddNTP to be added to the target nucleic acid.

Alternatively, an INVADER® assay can be used (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535). For example, an INVADER® operating system (OS), provides a method for detecting and quantifying DNA and RNA. The INVADER® OS is based on a “perfect match” enzyme-substrate reaction. The INVADER® OS uses proprietary CLEAVASE® enzymes (Third Wave Technologies, Inc (Madison, Wis.)), which recognize and cut only the specific structure formed during the INVADER® process which structure differs between the different alleles selected for detection, i.e. the disease-causing allele and the normal allele as well as between the different selected SNPs. Unlike the PCR-based methods, the INVADER® OS relies on linear amplification of the signal generated by the INVADER® process, rather than on exponential amplification of the target.

In the INVADER® process, two short DNA probes hybridize to the target to form a structure recognized by the CLEAVASE® enzyme. The enzyme then cuts one of the probes to release a short DNA “flap.” Each released flap binds to a fluorescently-labeled probe and forms another cleavage structure. When the CLEAVASE® enzyme cuts the labeled probe, the probe emits a detectable fluorescence signal.

Mutations or polymophisms may also be detected using allele-specific hybridization followed by a MALDI-TOF-MS detection of the different hybridization products. In the preferred embodiment, the detection of the enhanced or amplified nucleic acids representing the different alleles is performed using matrix-assisted laser desorption ionization/time-of-flight (MALDI-TOF) mass spectrometric (MS) analysis described in the Examples below. This method differentiates the alleles based on their different mass and can be applied to analyze the products from the various above-described primer-extension methods or the INVADER® process.

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotying method which involves the use of PCR amplification.

Computational algorithms such as expectation-maximization (EM), subtraction and PHASE are useful methods for statistical estimation of haplotypes (see, e.g., Clark, A. G. Inference of hapiotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

Genotyping CFH Alleles

According to one aspect of the present invention, a method for determining whether a human is homozygous for a polymorphism, heterozygous for a polymorphism, or lacking the polymorphism altogether (i.e. homozygous wildtype) is encompassed. For example, for the Tyr402His polymorphism, a method for determining the C-allele, heterozygous for the C- and T-alleles, or homozygous for the T-allele of the human CFH gene is provided. Substantially any method of detecting an allele of the CFH gene, such as hybridization, amplification, restriction enzyme digestion, and sequencing methods, can be used.

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotying method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

In one embodiment, an allelic discrimination method for identifying the CFH genotype of a human can be used. The allelic discrimination method of the invention involves use of a first oligonucleotide probe which anneals with a target portion of the individual's genome. The target portion comprises a portion of the region of CFH gene to be screened, for example, including the nucleotide residue at position 1277 in SEQ ID NO: 1. Because the nucleotide residue at this position differs, for example at position in the C-allele and the T-allele, the first probe is completely complementary to only one of the two alleles. Alternatively, a second oligonucleotide probe can also be used which is completely complementary to the target portion of the other of the two alleles. The allelic discrimination method of the invention also involves use of at least one, and preferably a pair of amplification primers for amplifying a reference region of the CFH gene of an individual. The reference region includes at least a portion of the human CFH, for example a portion including the nucleotide residue at position 1277 of the CFH gene in SEQ ID NO: 1.

Because the reference region and the target portion overlap by at least one base, preferably by at least about half the length of the target portion, and more preferably completely overlap, the enzyme (e.g. Thermus aquaticus {Taq} DNA polymerase) which catalyzes the amplification reaction and the first (or second) probe will collide. If the probe is not completely complementary to the target portion, it is more likely to dissociate from the target portion upon collision than if it is completely complementary. Therefore, unless the enzyme exhibits 5′→3′ exonuclease activity, amplification ceases or is greatly inhibited.

If the enzyme which catalyzes the amplification reaction exhibits 5′→3′ exonuclease activity (e.g. Taq DNA polymerase), then the enzyme will at least partially degrade the 5′-end of a probe with which it collides unless the probe dissociates from the target portion upon collision with the enzyme. As noted above, if the probe is not completely complementary to the target portion, it is much more likely to dissociate from the target portion upon collision than if it is completely complementary. If a detectable label is attached to a nucleotide residue at or near the 5′-end of the probe, release of the detectable label from the probe can be used as an indication that the enzyme and probe have collided and that the probe did not dissociate from the target portion. Thus, release of the detectable label from the probe upon amplification of the region indicates that the probe was completely complementary to the target portion. By selecting either or both of a probe completely complementary to the target portion of the polymorphism of the CFH gene and a probe completely complementary to the wildtype site at the target portion of the gene and assessing release of the label from the probe(s), the identity of the allele(s) can be ascertained.

The probe is preferably a DNA oligonucleotide having a length in the range from about 20 to about 40 nucleotide residues, preferably from about 20 to about 30 nucleotide residues, and more preferably having a length of about 25 nucleotide residues. In one embodiment, the probe is rendered incapable of extension by a PCR-catalyzing enzyme such as Taq polymerase, for example by having a fluorescent probe attached at one or both ends thereof. Although non-labeled oligonucleotide probes can be used in the kits and methods of the invention, the probes are preferably detectably labeled. Exemplary labels include radionuclides, light-absorbing chemical moieties (e.g. dyes), fluorescent moieties, and the like. Preferably, the label is a fluorescent moiety, such as 6-carboxyfluorescein (FAM), 6-carboxy-4,7,2′,7′-tetrachlorofluoroscein (TET), rhodamine, JOE (2,7-dimethoxy-4,5-dichloro-6-carboxyfluorescein), HEX (bexachloro-6-carboxyfluorescein), or VIC.

In a particularly preferred embodiment, the probe of the invention comprises both a fluorescent label and a fluorescence-quenching moiety such as 6-carboxy-N,N,N′,N′-tetramethylrhodamine (TAMRA), or 4-(4′-dimethlyaminophenylazo)benzoic acid (DABCYL). When the fluorescent label and the fluorescence-quenching moiety are attached to the same oligonucleotide and separated by no more than about 40 nucleotide residues, and preferably by no more than about 30 nucleotide residues, the fluorescent intensity of the fluorescent label is diminished. When one or both of the fluorescent label and the fluorescence-quenching moiety are separated from the oligonucleotide, the intensity of the fluorescent label is no longer diminished. Preferably, the probe of the invention has a fluorescent label attached at or near (i.e. within about 10 nucleotide residues of) one end of the probe and a fluorescence-quenching moiety attached at or near the other end. Degradation of the probe by a PCR-catalyzing enzyme releases at least one of the fluorescent label and the fluorescence-quenching moiety from the probe, thereby discontinuing fluorescence quenching and increasing the detectable intensity of the fluorescent labels. Thus, cleavage of the probe (which, as discussed above, is correlated with complete complementarity of the probe with the target portion) can be detected as an increase in fluorescence of the assay mixture.

If detectably different labels are used, more than one labeled probe can be used. For example, the assay mixture can contain a first probe which is completely complementary to the target portion of the polymorphism of the CFH gene and to which a first label is attached, and a second probe which is completely complementary to the target portion of the wildtype allele. When two probes are used, the probes are detectably different from each other, having, for example, detectably different size, absorbance, excitation, or emission spectra, radiative emission properties, or the like. For example, a first probe can be completely complementary to the target portion of the polymorphism and have FAM and TAMRA attached at or near opposite ends thereof. The first probe can be used in the method of the invention together with a second probe which is completely complementary to the target portion of the wildtype allele and has TET and TAMRA attached at or near opposite ends thereof. Fluorescent enhancement of FAM (i.e. effected by cessation of fluorescence quenching upon degradation of the first probe by Taq polymerase) can be detected at one wavelength (e.g. 518 nanometers), and fluorescent enhancement of TET (i.e. effected by cessation of fluorescence quenching upon degradation of the second probe by Taq polymerase) can be detected at a different wavelength (e.g. 582 nanometers).

Ideally, the probe exhibits a melting temperature (Tm) within the range from about 60° C. to 70° C., and more preferably in the range from 65° C. to 67° C. Furthermore, because each probe is completely complementary to only one of the alleles of the CFH gene, each probe will necessarily have at least one nucleotide residue which is not complementary to the corresponding residue of the other allele. This non-complementary nucleotide residue of the probe is preferably located near the midsection of the probe (i.e. within about the central third of the probe sequence) and is preferably approximately equidistant from the ends of the probe. Thus, for example, the probe which is completely complementary to the polymorphic allele of the human CFH gene can, for example, be completely complementary to nucleotide residues surrounding position 1277 of the polymorphic allele, as defined by the positions of SEQ ID NO: 1. For example, because the C- and T-alleles differ at position 1277, this probe will have a mismatched base pair nine nucleotide residues from one end when it is annealed with the corresponding target portion of the T-allele.

By way of example, labeled probes having the sequences of SEQ ID NOs: 3-4 can be used, in conjunction with labeled probes having the sequences of SEQ ID NOs: 5 and 6 in order to determine the allelic content of an individual (e.g. to assess whether the mammal comprises one or both of an C allele and a T allele of CFH at position 1277). For example, custom TaqMan SNP genotyping probes for each allele can be designed using Primer Express® v2.0 software (Applied Biosystems) using recommended guidelines. Successful discrimination of each allele can be verified using population control individuals. Genomic DNA (e.g. 20 ng) can be amplified according to assay recomendatons and genotyping analysis performed, as described in greater detail below.

The size of the reference portion which is amplified according to the allelic discrimination method of the invention is preferably not more than about 100 nucleotide residues. It is also preferred that the Tm for the amplified reference portion with the genomic DNA or fragment thereof be in the range from about 57° C. to 61° C., where possible.

It is understood that binding of the probe(s) and primers and that amplification of the reference portion of the CFH gene according to the allelic discrimination method of the invention will be affected by, among other factors, the concentration of Mg⁺⁺ in the assay mixture, the annealing and extension temperatures, and the amplification cycle times. Optimization of these factors requires merely routine experimentation which are well known to skilled artisans.

Another allelic discrimination method suitable for use in the present invention employs “molecular beacons”. Detailed description of this methodology can be found in Kostrikis et al., Science 1998;279:1228-1229, which is incorporated herein by reference.

The use of microarrays comprising a multiplicity of reference sequences is becoming increasingly common in the art. Accordingly, another aspect of the invention comprises a microarray having at least one oligonucleotide probe, as described above, appended thereon.

It is understood, however, that any method of ascertaining an allele of a gene can be used to assess the genotype of the CFH gene in a mammal. Thus, the invention includes known methods (both those described herein and those not explicitly described herein) and allelic discrimination methods which may be hereafter developed.

With reference to nucleic acids of the invention, the term “isolated nucleic acid” or “isolated polynucleotide” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote. With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide. The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and the method used. For example, for diagnostic applications, depending on the complexity of the target sequence, the oligonucleotide probe typically contains 15-25 or more nucleotides, although it may contain fewer nucleotides. The probes herein are selected to be substantially complementary to different strands of a particular target nucleic acid sequence. This means that the probes must be sufficiently complementary so as to be able to “specifically hybridize” or anneal with their respective target strands. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

In the context of this invention, the term “probe” refers to a molecule which can detectably distinguish between target molecules differing in structure (e.g. nucleic acid or protein sequence). Detection can be accomplished in a variety of different ways depending on the type of probe used and the type of target molecule. Thus, for example, detection may be based on discrimination on detection of specific binding. Examples of such specific binding include antibody binding and nucleic acid, antibody binding to protein, nucleic acid binding to nucleic acid, or aptamer binding to protein or nucleic acid. Thus, for example, probes can include enzyme substrates, antibodies and antibody fragments, and preferably nucleic acid hybridization probes.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

“Primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example. a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an anti-parallel fashion, at least one nucleotide residue of the first region is capable of base pairing with a residue of the second region. Preferably, the first region comprises a first portion and the second region comprises a second portion, whereby, when the first and second portions are arranged in an anti-parallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, a first region of an oligonucleotide “flanks” a second region of the oligonucleotide if the two regions are adjacent one another or if the two regions are separated by no more than about 1000 nucleotide residues, and-preferably no more than about 100 nucleotide residues.

A second set of primers is “nested” with respect to a first pair of primers if, after amplifying a nucleic acid using the first pair of primers, each of the second pair of primers anneals with the amplified nucleic acid, such that the amplified nucleic acid can be further amplified using the second pair of primers.

Nucleic acid molecules of the present invention may be prepared by two general methods: (1) Synthesis from appropriate nucleotide triphosphates, or (2) Isolation from biological sources. Both methods utilize protocols well known in the art.

The availability of nucleotide sequence information, such as a full length nucleic acid sequence having SEQ ID NO: 1, enables preparation of isolated nucleic acid molecules of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a 1.4 kb double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire 1.4 kb double-stranded molecule. A synthetic DNA molecule so constructed may then be cloned and amplified in an appropriate vector.

Nucleic acid sequences of the present invention may also be isolated from appropriate biological sources using methods known in the art.

Also contemplated with the scope of the present invention are vectors or plasmids containing the nucleic acid sequence of SEQ ID NO: 1, and host cells or animals containing such vectors or plasmids. Methods for constructing vectors or plasmids containing the nucleic acid sequence of SEQ ID NO: 1, and host cells or animals containing the same are within the ability of persons skilled in the art of molecular biology.

SNPs, Polymorphisms, and Alleles

The genomes of all organisms undergo spontaneous mutation in the course of their continuing evolution, generating variant forms of progenitor genetic sequences (Gusella, Ann. Rev. Biochem. 55, 831-854 (1986)). The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population. The SNP position (interchangeably referred to herein as SNP, SNP site, or SNP locus) is usually preceded by and followed by highly conserved sequences of the allele (e.g., sequences that vary in less than 1/100 or 1/1000 members of the populations). An individual may be homozygous or heterozygous for an allele at each SNP position. A SNP can, in some instances, be referred to as a “cSNP” to denote that the nucleotide sequence containing the SNP is an amino acid coding sequence.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel” (Weber et al., “Human diallelic insertion/deletion polymorphisms”, Am J Hum Genet October 2002;71(4):854-62).

A synonymous codon change, or silent mutation/SNP (the terms “SNP” and “mutation” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 July 2001).

Causative SNPs are those SNPs that produce alterations in gene expression or in the expression, structure, and/or function of a gene product, and therefore are most predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic disease. Examples of genes in which a SNP within a coding sequence causes a genetic disease include sickle cell anemia and cystic fibrosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the disease. These SNPs, although not causative, are nonetheless also useful for diagnostics, disease predisposition screening, and other uses.

An association study of a SNP and a specific disorder involves determining the presence or frequency of the SNP allele in biological samples from individuals with the disorder of interest, such as AMD, and comparing the information to that of controls (i.e., individuals who do not have the disorder; controls may be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

A SNP may be screened in diseased tissue samples or any biological sample obtained from a diseased individual, and compared to control samples, and selected for its increased (or decreased) occurrence in a specific pathological condition, such as pathologies related to AMD. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype. Association studies may be conducted within the general population and are not limited to studies performed on related individuals in affected families (linkage studies).

As used herein, a “biological sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent.

Clinical trials have shown that patient response to treatment with pharmaceuticals is oflen heterogeneous. There is a continuing need to improve pharmaceutical agent design and therapy. In that regard, SNPs can be used to identify patients most suited to therapy with particular pharmaceutical agents (this is often termed “pharmacogenomics”). Similarly, SNPs can be used to exclude patients from certain treatment due to the patient's increased likelihood of developing toxic side effects or their likelihood of not responding to the treatment. Pharmnacogenomics can also be used in pharmaceutical research to assist the drug development and selection process. (Linder et al. (1997), Clinical Chemistry, 43, 254; Marshall (1997), Nature Biotechnology, 15, 1249; International Pat. Application WO 97/40462, Spectra Biomedical; and Schafer et al. (1998), Nature Biotechnology, 16, 3).

Particular SNP alleles, sometimes referred to as polymorphisms or polymorphic alleles, of the present invention can be associated with either an increased risk of having or developing AMD, or a decreased risk of having or developing AMD. SNP alleles that are associated with a decreased risk of having or developing AMD may be referred to as “protective” alleles, and SNP alleles that are associated with an increased risk of having or developing AMD may be referred to as “susceptibility” alleles or “risk factors”. Thus, whereas certain SNPs (or their encoded products) can be assayed to determine whether an individual possesses a SNP allele that is indicative of an increased risk of having or developing AMD (i.e., a susceptibility allele), other SNPs (or their encoded products) can be assayed to determine whether an individual possesses a SNP allele that is indicative of a decreased risk of having or developing AMD (i.e., a protective allele). Similarly, particular SNP alleles of the present invention can be associated with either an increased or decreased likelihood of responding to a particular treatment or therapeutic compound, or an increased or decreased likelihood of experiencing toxic effects from a particular treatment or therapeutic compound. The term “altered” may be used herein to encompass either of these two possibilities (e.g., an increased or a decreased risk/likelihood).

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers, may be designed to hybridize to either strand and SNP genotyping methods disclosed herein may generally target either strand. Throughout the specification, in identifying a SNP position, reference is generally made to the protein-encoding strand, only for the purpose of convenience.

Assessment of Predisposition to AMD

The present invention also provides a method of assessing the relative susceptibility of a mammal (e.g. a human) to AMD. The “relative” susceptibility of a mammal to AMD refers to the fact that, among a population of individuals in a population at large, some individuals are more likely to develop AMD than others. This differential potential is attributable, at least in part to the genetic makeup of the individuals in the population.

In accordance with the present invention, it has been discovered that the presence of a certain polymorphism of the CFH gene is correlated with greater susceptibility to AMD in humans. Thus, the method of the invention for assessing the relative susceptibility of an individual to AMD comprises determining whether the individual comprises a polymorphism in the CFH gene. One particularly preferred polymorphism is a C-allele at position 1277 of the CFH gene.

In conjunction with the genotyping methods of the present invention, one can also determine the presence of other known risk factors in an individual. For example, risk factors for development of complications of AMD including cigarette smoking, lack of exercise, hypertension, obesity (2, 21), and increased serum CRP levels or decreased serum CFH levels (22-25). Further, drusen with terminal complement deposition indistinguishable from AMD were observed in eyes from patients with a kidney disease (membranoproliferative glomerulonephritis type 11) that can be caused by mutations in CFH (26, 27).

Methods of Treatment

The present invention also provides methods for treatment of a patient who has been determined to carry a variance in the human CFH gene, e.g., a pre-disposing allele to AMD. In one embodiment, the patient has not yet expressed any symptoms of ocular disease. In another embodiment, the patient expresses symptoms. Symptoms are known to those of skill in the art and may include blurred vision with the center of vision becoming blurred and the region growing larger as the disease progresses, straight lines may appear wavy and central vision loss can occur rapidly. It is recognized that no symptoms may be noticed if only one eye is affected.

Treatment can include prophylaxis, including agents which slow or prevent the progression of ocular disease such as AMD. In a preferred embodiment, the treatment includes any means to inhibits angiogenesis, such as, for example, angiogenesis inhibitor and more preferably, VEGF inhibitors. Such therapies may include laser based therapies to destroy blood vessels, such as photodynamic laser therapy,(Visudyne®) or pegaptanib sodium (Macugen®). More preferably, the therapy is an angiogenesis inhibitor, such as a VEGF inhibitor. One preferred VEGF inhibitor is ranibizumab (Lucentis™). Other angiogenesis inhibitors and/or VEGF inhibitors are known to those of skill in the art and are useful in the treatment methods of the present invention. Individuals diagnosed by the methods of the present invention as being susceptible to AMD may also wish to take vitamins and minerals such as, about 500 milligrams (mg) of vitamin C, about 400 international units (IU) of vitamin E, about 5 mg of beta-carotene, and about 80 mg of zinc oxide. Laser photocoagulation may also be useful in the treatment of individuals possessing a pre-disposing allele to AMD, especially in individuals where other symptoms are present. Other treatments encompassed by the present invention are Transpupillary Thermotherapy (TTT), Implantable Miniature Telescope (IMT), RHEO procedure, and the administration of anecortave acetate (Retaane®), Squalamine Lactate (EVIZON™), and Combretastatin.

The compounds used in connection with the treatment methods of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

The methods of the present invention allow for the early detection of individuals susceptible to ocular diseases and cardiovascular diseases such as AMD. Thus, treatment may be initiated early, e.g. before or at the beginning of the onset of symptoms. The dosage required at these early stages will be lower than those needed at later stages of disease where the symptoms are severe. Such dosages are known to those of skill in the art and can be determined by the physician in response to the particular patient.

Kits

The present invention also provides a kit for performing the instant method disclosed herein. The kit comprises a plurality of reagents useful for performing the disclosed methods, and optionally further comprises an instructional material which describes how the method is performed.

By way of example, an exemplary kit for performing the allelic discrimination method of the invention comprises: a) a first oligonucleotide probe which anneals specifically with a target portion of the mammal's genome, wherein the target portion includes the nucleotide residue located at a polymorphic position of SEQ ID NO: 1, such as position 1277, the probe comprising a fluorescent label and a fluorescence quencher attached to separate nucleotide residues thereof, and b) a primer for amplifying a reference portion of corresponding wildtype allele of the CFH gene, the reference portion including the corresponding non-polymorphic (or wildtype) nucleotide residue, as defined by the sequence of SEQ ID NO: 1.

The kit may further comprise a DNA polymerase having 5′→3′ exonuclease activity. The kit may also comprise a second oligonucleotide probe having a different annealing specificity than the first (e.g. wherein the first is completely complementary to the target portion of the C-allele at position 1277 of SEQ ID NO: 1 and the second is completely complementary to the target portion of the T-allele at position 1277 of SEQ ID NO:1), a second primer (e.g. such that this and the other primer can be used to amplify at least the target portion by a PCR), or both. The kit may comprise an instructional material which can, for example, describe performance of the allelic discrimination method, the association between the presence of the C-allele and carcinogenic susceptibility, or both.

In an alternative embodiment of the present invention, the kit comprises at least one, and preferably two molecular beacon probes, as described herein. When the kit comprises two molecular beacon probes, one is preferably specific for (i.e. completely complementary to a region including the polymorphic nucleotide residue of SEQ ID NO: 1, e.g. nucleotide 1277) the polymorphic allele of the CFH gene, and the other is specific for the non-polymorphic allele. This kit may further comprise an instructional material, including a publication, a recording, a diagram, or any other medium of expression which can be used to communicate the usefulness of the composition of the invention for performing a method of the invention or for associating the presence of a polymorphic allele of the CFH gene in an individual with susceptibility to AMD. The instructional material of the kit of the invention can, for example, be affixed to a container which contains a kit of the invention or be shipped together with a container which contains the kit. Alternatively, the instructional material can be shipped separately from the container with the intention that the instructional material and the kit be used cooperatively by the recipient.

Also provided by the present invention are kits for predicting the susceptibility of an individual to AMD according to the one or more of the methods of the invention. The kit comprises a plurality of reagents useful for performing one of the methods as described above, and optionally further comprises an instructional material which describes how the method is performed and the association between the presence of a polymorphic allele of CFH and AMD susceptibility.

Although the foregoing disclosure is principally directed to kits and methods which are applicable to human AMD, it will be understood by the skilled artisan that such methods and kits are generally applicable to mammals of all sorts. Modification, where necessary, of the kits and methods of the invention to conform to non-human AMD is well understood, and the ordinarily skilled veterinary worker can design and perform such modification with merely ordinary, if any, experimentation. Representative mammals include, for example, primates, cattle, pigs, horses, sheep, cats, and dogs.

Solid Supports

Solid supports containing oligonucleotide probes for identifying the alleles, including polymorphic alleles, of the present invention can be filters, polyvinyl chloride dishes, silicon or glass based chips, etc. Such wafers and hybridization methods are widely available, for example, those disclosed by Beattie (WO 95/11755). Any solid surface to which oligonucleotides can be bound, either directly or indirectly, either covalently or noncovalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, about 2, 10, 100, 1000 to 10,000; 100,000, 400,000 or 1,000,000 of such features on a single solid support. The solid support, or the area within which the probes are attached may be on the order of a square centimeter.

Oligonucleotide probe arrays can be made and used according to any techniques known in the art (see for example, Lockchart et al. (1996), Nat. Biotechnol. 14: 1675-1680; McGall et al. (1996), Proc. Nat. Acad. Sci. USA 93: 13555-13460). Such probe arrays may contain at least two or more oligonucleotides that are complementary to or hybridize to two or more of the SNPs described herein. Such arrays may also contain oligonucleotides that are complementary or hybridize to at least about 2, 3, 4, 5, 6, 7, 8, 9, 10, 20, 30, 50 or more SNPs described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al. (1992), U.S. Pat. No. 5,143,854; Fodor et al. (1998), U.S. Pat. No. 5,800,992; Chee et al. (1998), U.S. Pat. No. 5,837,832.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al., (1993). WO 93/09668. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Databases

The present invention includes databases containing information concerning polymorphic alleles associated with AMD, for instance, information concerning polymorphic allele frequency and strength of the association of the allele with AMD and the like. Databases may also contain information associated with a given polymorphism such as descriptive information about the probability of association of the polymorphism with AMD. Other information that may be included in the databases of the present invention include, but is not limited to, SNP sequence information, descriptive information concerning the clinical status of a tissue sample analyzed for SNP haplotype, or the subject from which the sample was derived. The database may be designed to include different parts, for instance a SNP frequency database and a SNP sequence database. Methods for the configuration and construction of databases are widely available, for instance, see Akerblom et al., (1999) U.S. Pat. No. 5,953,727, which is herein incorporated by reference in its entirety.

The databases of the invention may be linked to an outside or external database. In a preferred embodiment, the external database may be the HGBASE database maintained by the Karolinska Institute, The SNP Consortium (TSC) and/or the databases maintained by the National Center for Biotechnology Information (NCBI) such as GenBank.

Any appropriate computer platform may be used to perform the necessary comparisons between polymorphic allele frequency and associated disorder and any other information in the database or provided as an input. For example, a large number of computer workstations are available from a variety of manufacturers, such as those available from Silicon Graphics. Client-server environments, database servers and networks are also widely available and appropriate platforms for the databases of the invention.

The databases of the invention may also be used to present information identifying the polymorphic alleles in a subject and such a presentation may be used to predict the likelihood that the subject will develop AMD. Further, the databases of the present invention may comprise information relating to the expression level of one or more of the genes associated with the polymorphic alleles of the invention.

The polymorphisms identified by the present invention may be used to analyze the expression pattern of an associated gene and the expression pattern correlated to the probability of developing an AMD. The expression pattern in various tissues can be determined and used to identify tissue specific expression patterns, temporal expression patterns and expression patterns induced by various external stimuli such as chemicals or electromagnetic radiation.

The following examples are provided to illustrate certain embodiments of the invention. They are not intended to limit in any way the remainder of the disclosure.

### EXAMPLES

Complement Factor H Polymorphism and Age-Related Macular Degeneration

Age-related macular degeneration (AMD) is a leading cause of blindness in older individuals (1). It is a late-onset, complex trait with hereditary, lifestyle, and medical risk factors (2). The condition typically presents in the fifth decade of life with small yellow deposits external to the outer retina and retinal pigment epithelium (RPE) called drusen. Large numbers of drusen and clinical features of damage to the RPE markedly increase the-risk of complications (atrophy of the RPE and abnormal neovascularization of the outer retina), leading to severe vision loss (1).

Although the primary pathogenic mechanisms of AMD were previously unknown, there is strong evidence that genetics plays a role (3-9). The first locus for AMD (ARMD1) was reported in a single extended family linked to chromosome 1q25.3-31.3 (5). Because there was strong evidence for linkage to this region of chromosome 1 from subsequently reported small family studies, we focused our efforts on the ARMD1 locus (3, 4, 6, 8, 9).

Source of Research Subjects:

Cases were patients of one of the authors (A.O.E.) and were prospectively and consecutively ascertained under a protocol approved by the University of Texas Southwestern (UTSW) Medical Center Institutional Review Board. All patients with any stage of age-related macular generation (AMD) were invited to participate by providing health information, diagnostic documentation, and a blood sample. Only patients unable to provide a blood sample and those who refused to participate were excluded. Patients were seen for management and follow up regardless of severity of disease, manifestations of disease, or presence of complications. The data are derived from a subpopulation of case and control individuals of European descent who met certain diagnostic criteria set forth below.

Controls were primarily patients of A.O.E. and other UTSW physicians and were over the age of 50 without a family history of AMD. Spouses of AMD patients were also prospectively recruited and spouses of UTSW patients were enrolled when possible. The spouses often obtained their medical care at UTSW Medical Center. Thus, the controls were selected from the same pool of patients receiving medical care at UTSW Medical Center as the cases. We attempted to obtain controls with a similar age distribution as the cases by ascertaining spouses and recruiting subjects age 60 or older. Potential controls were recruited for a screening examination. If the potential control subject met certain diagnostic criteria set forth below, fundus photography and full enrollment were performed.

Examination of Research Subjects:

Cases had a comprehensive ocular examination as a part of their regular medical care. Color fundus photographs were performed as indicated for their regular medical care or specifically for the study.

Potential control subjects were screened using a brief questionnaire to exclude individuals with a family history of AMD or ocular diseases that might simulate AMD or preclude its diagnosis such as prior laser photocoagulation, cryopexy, media opacity, and inflammatory diseases. Screened subjects were scheduled for a limited research eye examination that included visual acuity with correction, slit lamp evaluation of the anterior segment and vitreous, and biomicroscopy of the optic nerve head and macula. Potential control subjects were excluded for media opacity (e.g., cataract) preventing excellent visualization of the macula, diseases with phenotypic overlap with AMD such as drusen or pigmentary disturbance of the RPE insufficient to diagnose AMD, pattern dystrophies, toxoplasmosis, histoplasmosis, high myopia, central serous chorioretinopathy, or any disease or treatment that would diminish the ability to recognize drusen such as laser photocoagulation, macula involving retinal detachment, posterior uveitis, and trauma. Inclusion criteria were less than five small (<63 micron) hard drusen, at least 20/40 view of the fundus, and absence of exclusion criteria. Fundus photographs and a blood sample were taken on subjects meeting these criteria by eye examination. The examination findings for both cases and controls were systematically documented and a tentative diagnosis assigned in the clinic after the examination.

Diagnosis of Research Subjects:

The fundus photographs and examination notes were reviewed prior to the selection of case and control subjects for genotyping (Table 2). Discrepancies between the examination and fundus photographs were resolved by reevaluating the subject or selecting the more conservative diagnostic evaluation. Thus, features noted on examination but not visible on fundus photography were used to exclude control subjects but not to enroll AMD subjects. The medical records, examination notes, and fundus photographs were reviewed as collected and at least one additional time prior to selecting the subject for genotyping.

The AMD cases had one or more drusen ≧63 microns in diameter documented at some point during their disease course (S1). The minimum disease severity required for inclusion was high risk AMD defined as sufficient drusen in the macula to fill a circle 700 microns in diameter or drusen with more advanced features such as retinal pigment epithelial hyperplasia. The presence of these features is predictive of significant risk for developing complications of AMD such as exudation and has been used previously in AMD genetics studies (S2-4). Cases were diagnosed as (i) high risk, early AMD, (ii) AMD with pure geographic atrophy, or (iii) AMD with exudation using standard criteria (S1).

Hard drusen are common in subjects over the age of 50 (S5). The Beaver Dam Eye Study longitudinal data and our own cross-sectional observations with families segregating AMD support extensive hard drusen as a precursor to larger drusen and pigmentary abnormalities diagnostic of AMD (S6). Eight or more hard drusen doubled the 10-year incidence of AMD (S6). As noted above, we chose a conservative criterion of less than 5 hard drusen and the absence of more advanced features for enrollment of control subjects.

Disease Features of AMD Subjects:

A total of 400 AMD subjects were included in this study. The distribution of subjects was: 47% high risk, early AMD; 16% drusen-associated AMD complicated by pure geographic atrophy; and 37% drusen-associated AMD complicated by exudation. Patients with exudation and geographic atrophy are referred to as late AMD. Thus, 53% of subjects had late AMD.

Selection of Samples for Genotyping:

A subset of AMD subjects was selected for initial genotyping and designated the discovery sample. This sample preferentially included 83 probands with a family history consistent with dominant transmission of AMD toward the goal of potentially increasing our power to detect a genetic effect. The discovery sample preferentially included 119 subjects with high-risk, early AMD because these subjects were closer in age to the control population. The remaining subjects in the discovery sample were exudative with a family history of AMD that was not consistent with dominant transmission. A total of 224 AMD subjects and 134 control subjects made up the discovery sample.

The remainder of the subjects already available at the time of the selection of the discovery sample (161 subjects) and subsequently ascertained subjects (244 subjects) were prospectively utilized as a replication sample.

Genotyping

SNP Selection, Assay Design, and Validation:

All SNPs intended for genotyping were selected from the public domain, as reported by www.ensembl.org and http://www.ncbi.nlm.nih.gov/SNP/snpLocus.html. SNPs with known allele frequencies were selected on the basis of conferring amino acid substitutions in encoded proteins (non-synonymous coding SNPs) or for approximately equal spacing throughout genes (gene-based SNPs).

Genotyping was performed using either TaqMan® SNP genotyping assays (Applied Biosystems, Foster City, Calif.) or the MassARRAY® platform (SEQUENOM, San Diego, Calif.). Custom TaqMan SNP genotyping probes (S7) for each allele were designed using Primer Express® v2.0 software (Applied Biosystems) using recommended guidelines (S8). Successful discrimination of each allele was verified using 31 Caucasian population control individuals. Genomic DNA (20 ng) was amplified according to assay recommendations and genotyping analysis was performed using an ABI Prism® 7900HT. Assays that failed to discriminate alleles from any control population sample were either redesigned and retested or not used for further genotyping analysis.

Alternatively, sequences containing SNPs were used in multiplex assay designs using proprietary software SpectroDESIGNER®. Designed assays were validated using 25 ng of a genomic DNA pool containing an equimolar mix of DNA from 92 unrelated individuals of European ancestry derived from the Centre d'Etude du Polymorphisme Humaine (Paris) panel (S9, 10). Assays were analyzed by MALDI-TOF mass spectrometry on the MassARRAY platform, and allele frequencies within the DNA pool were estimated as described (S9). Working assays for SNPs with minor allele frequencies estimated at approximately 0.1 and higher were employed for all SNP assays presented in this manuscript.

Genotyping of Discovery and Replication Samples

TaqMan SNP assays for genotyping were performed in the same manner as for validating the assays. For the MassARRAY platformn, genotyping was performed as described (S9, 11-14). Briefly, sequences containing target SNPs were amplified from 2.5 ng genomic DNA in reactions including from one to six multiplexed assays. Unincorporated dNTPs following PCR were eliminated using shrimp alkaline phosphatase. Amplification products were then used in MassEXTEND® reactions to generate allele-specific products. EXTEND oligonucleotides annealing immediately adjacent to amplified target SNPs were extended using a DNA polymerase in the presence of a dNTP/ddNTP mix to generate primer extension products differing in length and mass as specified by the SNP alleles. Following brief treatment with resin to desalt the reactions, aliquots of EXTEND reaction products were deposited onto 384-array microchips and analyzed by MALDI-TOF mass spectrometry. Individual mass spectra were processed in real-time by proprietary SpectroTYPER software using baseline correction and peak identification algorithms to determine alleles based on input assay design parameters. Quality control of genotype calls was then performed post-real time using proprietary GenoQC software, which plots individual calls on an X-Y grid and evaluates the statistical likelihood of each call belonging to the genotype class to which it was assigned in real-time.

Statistical Analyses

Subject Demographics

Differences in mean age and presence or absence of family history in the discovery and replication samples were evaluated using the t-test.

Hardy-Weinberg Equilibrium

Hardy-Weinberg equilibrium (HWE) implies that in a sample of subjects selected from a larger population, the SNP genotype frequencies can be predicted from the allele frequencies. Specifically, if ‘p’ is the population frequency of allele ‘A’ and ‘q’ is the population frequency of allele ‘B’, the frequency of the genotypes, AA, AB, and BB should fit the distribution of p²+2pq,+q²=1 to be in HWE (S15). All SNPs reported in this study met HWE conditions.

Allele and Genotype Analyses

Allele and genotype frequency distributions were compared between cases and controls using a χ² goodness-of-fit test. Because the cases were significantly older than controls at the time of entry into the study, these analyses were repeated using logistic regression models adjusting for age. However, this adjustment did not change the significance level or patterns of association. Analyses were carried out using the SAS System, Release 8.02, SAS Institute Inc, Cary, N.C., 2002.

Haplotype Analysis

We examined haplotype block structures and generated haplotypes in these blocks using the Haploview software (S16). This approach assumes that all subjects are unrelated and that all haplotypes are ambiguous due to unknown linkage phase of the SNPs. The resultant block structure was determined using the algorithm of Gabriel et al. (S17). Within each block, at least 95% of informative pair-wise SNP comparisons show strong LD (i.e., the LD statistic, D′(S15), is 0.8 or greater). Comparisons of marker haplotypes with AMD were carried out using the haplo.score function of the Haplo.Stats program (ver 1.1.1) by D. Schaid, J. Sinnwell, C. Rowland, D. Tines (Mayo Clinic) web: http://www.mayo.edu/hsr/people/schaid.html; and Haploview 3.0 by J. Barrett (Whitehead Institute) web: httip://www.broad.mit.edu/personal/icbarret/haploview/. The resultant statistic for the global test of association fits an asymptotic χ² distribution. Comparison were made to the HapMap data (S18).

Attributable Risk

The measurement of attributable fraction (AF) for the CC and CT genotypes at Try402His was calculated using formulas which consider the frequency of CC and CT in the cases:

Error! Objects cannot be created from editing field codes. or controls: Error! Objects cannot be created from editing field codes. where f is the fraction of cases or controls with CC and CT, and R is the measure of relative risk. For more information on attributable fraction for genetic traits, see Khoury M J, Beaty T H, Cohen B H. Fundamentals of Genetic Epidemiology. New York, Oxford University Press, 1993, pp. 77-79.

Evaluation of the Fibulin 6 Gene

A rare Q5346R variation in a conserved amino acid of the Fibulin 6 gene (FIBL6) was proposed as the mutation causing AMD in the original family (AMD30) defining the ARMD1 locus (S19). Even though this protein is a candidate gene for AMD based on homology to an early-onset hereditary macular disease characterized by dominant radial drusen, the variation was not associated with AMD in multiple studies (S4, 19-24). We observed a single case (224 genotyped subjects) and a single control (134-genotyped subjects) with the Q5346R variation giving a combined frequency of 0.0056.

While the Q5346R variation cannot account for the observed linkage to 1q25.3-31.3 in small family studies, the possibility of other variation in FIBL6 contributing to the linkage signal has not been excluded. To address this possibility, we performed allele association studies by genotyping 23 SNPs across FIBL6 spaced at an average density of 20 kb in the discovery sample (Table 4). The individual polymorphisms and haplotypes derived from them were not associated with AMD. Thus, genetic variation across the 456 kb FIBL6 locus is

unlikely to contribute to the linkage signal on chromosome 1q25.3-31.3 and to AMD susceptibility in our case-control sample.

Results

We performed an allele association study on a new case-control population highly discordant for clinical phenotypes. Cases were enrolled based on ocular features (extensive drusen or pigmentary abnormalities of the macula) placing subjects at high risk for development of the complications of AMD or the presence of those complications in one or both eyes (10). Control subjects were from the same patient population and could have no more than four small hard drusen in the central retina (macula) and no known family history of AMD. A subset of 224 cases and 134 controls meeting these criteria were selected as a discovery sample for initial genotyping (Table 2). The discovery sample was enriched for AMD cases showing familial clustering of AMD and high-risk, early AMD. A second, replication sample of 176 cases and 68 controls was ascertained at the same clinic following the same protocol (Table 2).

Evaluation of the reported Q5346R variation in the Fibulin 6 gene (FIBL6) and 23 SNPs across this gene (supporting online text) gave no evidence for allele or haplotype association in the discovery sample (11). To determine if common coding sequence variation within the ARMD1 locus was associated with AMD, we searched publicly available databases for non-synonymous coding SNPs (nscSNPs). We identified 24 nscSNPs with known minor allele frequency of at least 10% across the 14 Mb ARMD1 locus. Genotyping of the discovery sample gave significant allele and genotype association between AMD and nscSNPs only within the regulation of complement activation (RCA) locus in chromosome 1q31.3 (Table 3). Additional evenly spaced, gene-based SNPs were evaluated across all 31 genes in clusters 1, 3, 4, and 6 at 8 kb to 25 kb density (FIG. S1) and no associations with AMD were detected outside of the RCA locus (Table 4). These data suggest the RCA locus contains one or more genetic variants increasing the risk of developing AMD.

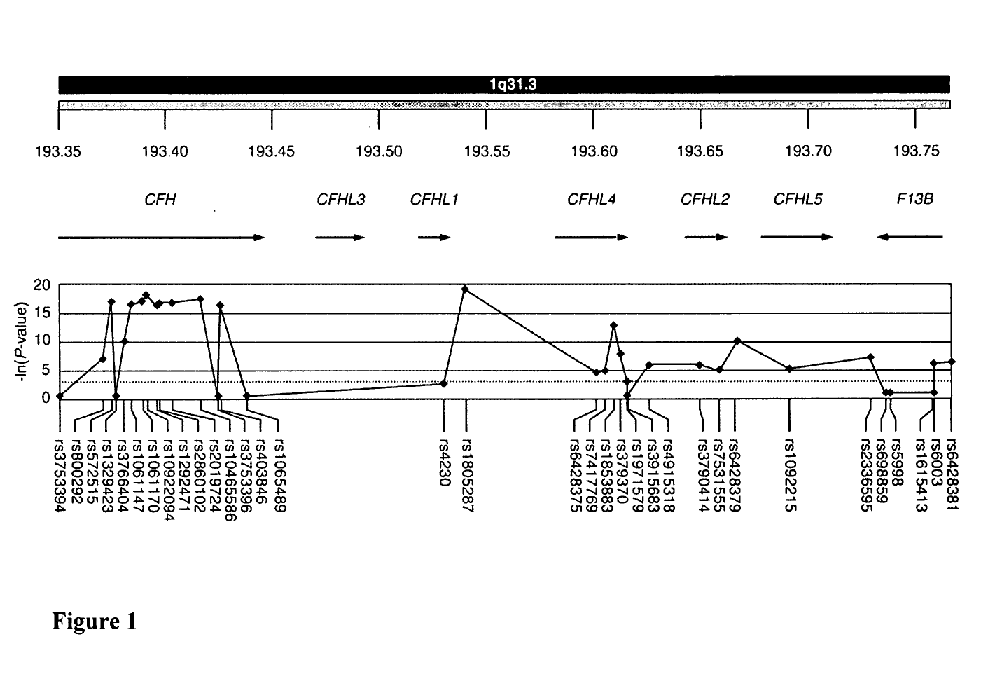

The RCA locus spans 388 kb of genomic DNA that contains the gene encoding complement factor H (CFH), five genes derived from CFH through ancestral duplications, and the gene encoding factor 13B (FIG. 1). A total of 86 SNPs located across the RCA locus and flanking regions were genotyped. Twenty-nine gave evidence for allele association with the majority and most significant of these, including the nscSNP rs1061170, concentrated in the CFH gene (Table 4, GenBank accession NM_{—}000186).

The genotype frequency data for rs lO61170 revealed that the association with AMD was largely due to an excess of CC homozygotes in cases compared to controls (Table 1). A similar pattern of association was evident with seven adjacent SNPs in CFH (FIG. 1 and Table 4). The strength of the evidence for association diminished markedly with SNPs located immediately proximal to CFH and distal to its derivatives CFHL1-CFHL5, suggesting that the effect was due to one or more polymorphisms in the complement factor genes only (FIG. 1 and Table 4).

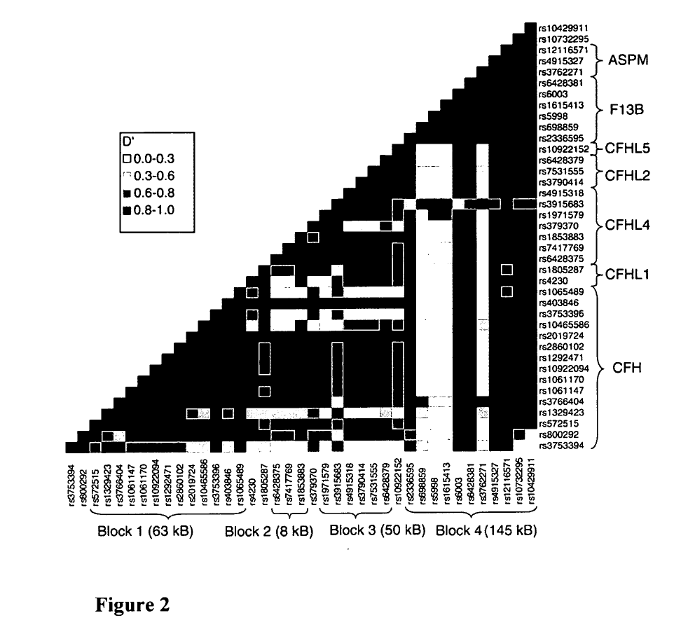

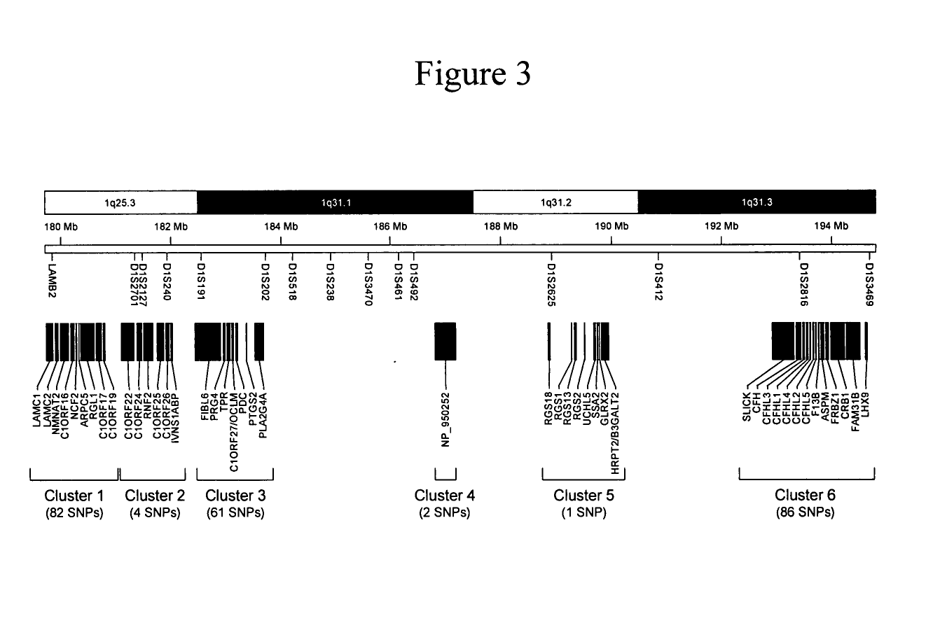

Haplotype analyses of 34 SNPs spanning 418 kb revealed extensive linkage disequilibrium across the full length of the RCA locus (FIG. 2). The highest level of linkage disequilibrium discernable among 4 haplotype blocks was across the RCA locus. Thirteen contiguous SNPs in CFH (i.e., all but the first two CFH SNPs in FIG. 2) form a 64 kb haplotype block. A second 9 kb haplotype block contained SNPs in the proximal portion of CFHL4, a third 50 kb block contained SNPs in the distal portion of CFHL4 and SNPs in CFHL2, and a fourth 146 kb block contained SNPs in F13B, ASPM and FRBZ1 (FIG. 2). The SNPs most significantly associated with AMD were in CFH or within 221 kb downstream of CFH (FIG. 1 and Table 4).

The association between AMD and haplotypes comprising 2-5 contiguous SNPs was evaluated using the Haplo.stat software by a sliding window approach (12, 13). Additional comparisons were made using all possible haplotypes formed by pair wise combinations including at least one nscSNP within the RCA locus. The analysis did not reveal any SNP combination showing greater association with AMD than the individual SNPs. All of the AMD high-risk haplotypes including a CFH SNP from haplotype block 1 and a non-CFH SNP from other regions of the RCA locus (FIG. 1) contained the AMD risk allele from the CFH SNP but not necessarily the AMD risk allele from the non-CFH SNP. These analyses provide further evidence that the multiple signals in the RCA locus are related to a single haplotype and therefore likely caused by a single genetic effect.

To verify these findings, we genotyped 14 SNPs in the RCA locus in the replication sample. Association with AMD was observed with the 7 markers that were significant in the discovery sample, including rs1061170, but not with the 7 markers yielding negative results in the discovery sample (Table 5). Notably, the genotype frequencies for rs1061170 among cases enriched for a positive family history of AMD were nearly identical to the frequencies among cases without this characteristic (Table 1), suggesting that this CFH protein polymorphism is a risk factor for AMD more generally. Taking into account data from the entire sample, a conservative estimation of the relative risk for AMD conferred by having at least one C allele (i.e., having either the CC or CT genotypes) was 2.7 (95% CI=1.9-3.9).

Complement activation has been implicated in the pathogenesis of a number of complex traits including AMD and can arise through the classical, lectin, or alternative pathways (14). All three pathways lead to the generation of a C3 convertase enzyme and subsequent activation of the immune response, the terminal pathway pore-like membrane attack complex (C5b-9), and cell lysis. The alternative complement pathway is spontaneously activated and CFH is an essential inhibitor preventing uncontrolled complement activation (15). Components of the terminal complement pathway and other markers of inflammation are deposited in drusen and the choroid of eyes with AMD (16, 17). Abnormal regulation of the alternative pathway of complement activation by CFH is consistent with these observations.

The tyrosine to histidine polymorphism (rs 1061170) at amino acid 402 of CFH appears to be a primary pathogenic variation increasing the risk of developing AMD. CFH is composed of 20 repetitive units of 60 amino acids called short consensus repeats (SCRs). The Try402His polymorphism is located within SCR7, which contains the overlapping binding sites for heparin, C-reactive protein (CRP), and M-protein (18). Serum levels of CRP were elevated in AMD subjects compared to controls in one large prospective clinical trial (19). CRP activates the classic complement pathway, but reduces deposition of C5b-9 through the direct binding of CFH (20). Risk factors for development of complications of AMD including cigarette smoking, lack of exercise, hypertension, and obesity (2, 21) increased serum CRP levels or decreased serum CFH levels (22-25). Further, drusen with terminal complement deposition indistinguishable from AMD were observed in eyes from patients with a kidney disease (membranoproliferative glomerulonephritis type II) that can be caused by mutations in CFH (26, 27). In principle, altered binding of CFH to CRP or heparin on outer retinal surfaces caused by the Tyr402His substitution could affect the level of inflammation in the outer retina, thereby contributing to AMD. Although our results are consistent with the Tyr402His variant causing AMD, they do not rule out the existence of other coding or splice site variants within CFH that modulate risk of AMD.

More than 7 million individuals in the United States have retinal features placing them at high risk for developing vision loss from complications of AMD (28). The attributable fraction for the C allele derived from the total sample of subjects in this study is 50%, indicating that persons either homozygous or heterozygous for histidine at amino acid 402 of CFH account for one-half of AMD cases. Given the rapid aging of the population, an estimated 3 million individuals will have atrophic and exudative complications of AMD by 2020 (28).

TABLE 1
Association between the Try402His polymorphism (rs1061170) in CFH and AMD.
The C allele codes for histidine. The genotype association compares CC to CT and TT.

| | Allele | | | Allele | Genotype | | | Genotype |
|---|---|---|---|---|---|---|---|---|
| | distribution | | | Association | distribution | | | Association |
| Sample | | Cases | Controls | (P-value) | | Cases | Controls | (P-value) |
| Discovery | C | 0.553 | 0.340 | 3.68 × 10⁻⁸ | CC | 0.320 | 0.115 | 7.67 × 10⁻⁷ |
| | T | 0.447 | 0.660 | | TC | 0.467 | 0.450 | |
| | | | | | TT | 0.213 | 0.435 | |
| Replication | C | 0.544 | 0.390 | 0.0039 | CC | 0.306 | 0.186 | 0.0135 |
| | T | 0.456 | 0.610 | | TC | 0.477 | 0.407 | |
| | | | | | TT | 0.218 | 0.407 | |
| Total | C | 0.549 | 0.355 | 4.95 × 10⁻¹⁰ | CC | 0.314 | 0.137 | 1.4 × 10⁻⁸ |
| | T | 0.451 | 0.645 | | TC | 0.471 | 0.437 | |
| | | | | | TT | 0.215 | 0.426 | |

TABLE 2
Demographics of the Discovery and Replication Case-Control Samples

| | Number of | Percent | Mean Age at | Percent with Family |
|---|---|---|---|---|
| Sample | subjects | Male | Entry (SD) | History of AMD |
| Discovery | | | | |
| AMD Cases | 224 | 58 | 72.7 (10.1) | 46.7 |
| Controls | 134 | 42 | 67.6 (7.6) | 0 |
| Replication* | | | | |
| AMD Cases | 176 | 65 | 78.2 (7.9) | 18.8 |
| Controls | 68 | 35 | 68.1 (9.0) | 0 |

*As expected from the selection criteria, the cases in the replication sample had a lower percentage of family history of AMD (P = 1.0 × 10⁻¹²) and an older age at entry into the study (P = 2.7 × 10⁻⁹) compared with cases in the discovery sample

TABLE 3
Allele and genotype association between AMD and polymorphic
non-synonymous coding SNPs (nscSNPs) in the 14 Mb ARMD1 locus.

| | | | | P-value for | |
|---|---|---|---|---|---|
| | | | | Association | |
| | | | | with AMD** | |
| Gene | Cluster* | nscSNP | Map Position | Allele | Genotype |
| LAMC2 | 1 | rs11586699 | 179916347 | 0.26 | 0.42 |
| LAMC2 | 1 | rs2296306 | 179922981 | 0.62 | 0.62 |
| LAMC2 | 1 | rs2296303 | 179933627 | 0.22 | 0.45 |
| C1orf16 | 1 | rs2298083 | 180247085 | 0.43 | 0.59 |
| NCF2 | 1 | rs2274064 | 180274044 | 0.32 | 0.16 |
| MGC26594 | 1 | rs1174657 | 180348583 | 0.51 | 0.78 |
| MGC26594 | 1 | rs1174658 | 180348762 | 0.76 | 0.91 |
| C1orf19 | 1 | rs1046934 | 180755186 | 0.40 | 0.35 |
| C1orf22 | 2 | rs9425343 | 181395194 | 0.99 | 0.99 |
| C1orf26 | 2 | rs10489579 | 181875378 | 0.34 | 0.54 |
| C1orf26 | 2 | rs6698109 | 181903526 | 0.99 | 0.79 |
| C1orf26 | 2 | rs12041704 | 181972131 | 0.25 | 0.46 |
| FIBL6 | 3 | rs7539719 | 182690394 | 0.38 | 0.12 |
| PRG4 | 3 | rs2273779 | 183005651 | 0.88 | 0.98 |
| TPR | 3 | rs3753565 | 183048145 | 0.35 | 0.64 |
| C1orf27 | 3 | rs12084264 | 183094776 | 0.99 | 1.0 |
| GLRX2 | 5 | rs10921310 | 189806308 | 0.09 | 0.11 |
| CFH | 6 | rs800292 | 193373890 | 0.0009 | 0.0022 |
| CFH | 6 | rs1061170 | 193390894 | 3.68 × | 7.67 × |
| | | | | 10⁻⁸ | 10⁻⁷ |
| CFH | 6 | rs1065489 | 193441431 | 0.98 | 0.69 |
| CFHL4 | 6 | rs379370 | 193615850 | 0.0004 | 0.0012 |
| F13B | 6 | rs6003 | 193762678 | 0.0029 | 0.0038 |
| ASPM | 6 | rs3762271 | 193802099 | 0.56 | 0.21 |
| ASPM | 6 | rs12138336 | 193802178 | 0.80 | 0.42 |

*Please refer to FIG. 1,
**All SNPs presented in this manuscript were in Hardy-Weinberg equilibrium. Additional genotype data is presented in Table 4.

TABLE 4
Genotyping in the discovery sample: Allele and genotype association between AMD
and all 236 polymorphic SNPs across gene clusters 1-6 of the ARMD1 locus.*

| | | | | | | χ²1 df | | | | χ²2 df |
|---|---|---|---|---|---|---|---|---|---|---|
| | | | Allele Counts | | | Test | Genotype Counts | | | Test |
| Cluster | SNP Name | Location | | AMD | Control | p-value | | AMD | Control | p-value |
| 1 | rs4593781 | 179716640 | C | 195 | 129 | 0.25 | CC | 40 | 29 | 0.50 |
| | | | T | 251 | 139 | | CT | 115 | 71 | |
| | | | | | | | TT | 68 | 34 | |
| 1 | rs4129857 | 179736204 | A | 237 | 135 | 0.34 | AA | 63 | 33 | 0.62 |
| | | | G | 189 | 125 | | AG | 111 | 69 | |
| | | | | | | | GG | 39 | 28 | |
| 1 | rs6664995 | 179749002 | C | 248 | 138 | 0.35 | CC | 66 | 34 | 0.63 |
| | | | T | 196 | 126 | | CT | 116 | 70 | |
| | | | | | | | TT | 40 | 28 | |
| 1 | rs7547953 | 179763298 | G | 250 | 140 | 0.29 | GG | 67 | 34 | 0.55 |
| | | | T | 194 | 128 | | GT | 116 | 72 | |
| | | | | | | | TT | 39 | 28 | |
| 1 | rs4397624 | 179784788 | C | 188 | 122 | 0.49 | CC | 38 | 26 | 0.76 |
| | | | T | 234 | 136 | | TC | 112 | 70 | |
| | | | | | | | TT | 61 | 33 | |
| 1 | rs7542640 | 179795395 | A | 255 | 139 | 0.23 | AA | 71 | 35 | 0.47 |
| | | | G | 187 | 123 | | GA | 113 | 69 | |
| | | | | | | | GG | 37 | 27 | |
| 1 | rs1537520 | 179815886 | C | 184 | 116 | 0.42 | CC | 37 | 24 | 0.68 |
| | | | T | 252 | 140 | | TC | 110 | 68 | |
| | | | | | | | TT | 71 | 36 | |
| 1 | rs1886499 | 179826681 | C | 192 | 125 | 0.32 | CC | 39 | 27 | 0.59 |
| | | | T | 256 | 143 | | TC | 114 | 71 | |
| | | | | | | | TT | 71 | 36 | |
| 1 | rs3768616 | 179835208 | A | 182 | 119 | 0.20 | AA | 36 | 26 | 0.41 |
| | | | G | 250 | 133 | | AG | 110 | 67 | |
| | | | | | | | GG | 70 | 33 | |
| 1 | rs729672 | 179840893 | C | 185 | 121 | 0.36 | CC | 39 | 28 | 0.65 |
| | | | T | 235 | 133 | | CT | 107 | 65 | |
| | | | | | | | TT | 64 | 34 | |
| 1 | rs7473 | 179846291 | A | 250 | 140 | 0.35 | AA | 69 | 36 | 0.63 |
| | | | G | 188 | 122 | | GA | 112 | 68 | |
| | | | | | | | GG | 38 | 27 | |
| 1 | rs7513264 | 179880946 | C | 221 | 136 | 0.80 | CC | 58 | 37 | 0.95 |
| | | | G | 203 | 120 | | CG | 105 | 62 | |
| | | | | | | | GG | 49 | 29 | |
| 1 | rs544352 | 179889203 | A | 228 | 130 | 0.58 | AA | 60 | 31 | 0.76 |
| | | | G | 206 | 128 | | AG | 108 | 68 | |
| | | | | | | | GG | 49 | 30 | |
| 1 | rs552361 | 179899515 | C | 283 | 168 | 0.80 | CC | 86 | 57 | 0.30 |
| | | | T | 165 | 94 | | TC | 111 | 54 | |
| | | | | | | | TT | 27 | 20 | |
| 1 | rs635796 | 179909111 | C | 242 | 157 | 0.27 | CC | 68 | 48 | 0.54 |
| | | | T | 200 | 109 | | TC | 106 | 61 | |
| | | | | | | | TT | 47 | 24 | |
| 1 | rs11586699 | 179916347 | C | 409 | 250 | 0.26 | CC | 190 | 118 | 0.42 |
| | | | T | 33 | 14 | | CT | 29 | 14 | |
| | | | | | | | TT | 2 | 0 | |
| 1 | rs3814337 | 179919719 | C | 347 | 194 | 0.30 | CC | 137 | 75 | 0.40 |
| | | | T | 85 | 58 | | TC | 73 | 44 | |
| | | | | | | | TT | 6 | 7 | |
| 1 | rs2296303 | 179933627 | C | 68 | 50 | 0.22 | CC | 4 | 4 | 0.45 |
| | | | G | 382 | 218 | | CG | 60 | 42 | |
| | | | | | | | GG | 161 | 88 | |
| 1 | rs1925043 | 179939260 | A | 343 | 198 | 0.46 | AA | 137 | 79 | 0.45 |
| | | | G | 81 | 54 | | AG | 69 | 40 | |
| | | | | | | | GG | 6 | 7 | |
| 1 | rs649954 | 179948105 | C | 357 | 207 | 0.60 | CC | 141 | 84 | 0.23 |
| | | | T | 89 | 57 | | TC | 75 | 39 | |
| | | | | | | | TT | 7 | 9 | |
| 1 | rs2105160 | 179957786 | C | 87 | 56 | 0.62 | CC | 6 | 8 | 0.26 |
| | | | G | 359 | 210 | | CG | 75 | 40 | |
| | | | | | | | GG | 142 | 85 | |
| 1 | rs2301877 | 179990782 | C | 274 | 160 | 0.35 | CC | 82 | 56 | 0.17 |
| | | | T | 172 | 86 | | CT | 110 | 48 | |
| | | | | | | | TT | 31 | 19 | |
| 1 | rs7521935 | 180001747 | C | 322 | 191 | 0.88 | CC | 116 | 68 | 0.98 |
| | | | G | 120 | 73 | | GC | 90 | 55 | |
| | | | | | | | GG | 15 | 9 | |
| 1 | rs951420 | 180011569 | A | 74 | 56 | 0.11 | AA | 4 | 6 | 0.20 |
| | | | G | 372 | 206 | | GA | 66 | 44 | |
| | | | | | | | GG | 153 | 81 | |
| 1 | rs1411392 | 180021707 | C | 142 | 71 | 0.14 | CC | 21 | 5 | 0.13 |
| | | | T | 300 | 193 | | CT | 100 | 61 | |
| | | | | | | | TT | 100 | 66 | |
| 1 | rs953274 | 180031538 | C | 205 | 125 | 0.99 | CC | 43 | 29 | 0.75 |
| | | | T | 225 | 137 | | TC | 119 | 67 | |
| | | | | | | | TT | 53 | 35 | |
| 1 | rs1541170 | 180041475 | C | 298 | 191 | 0.17 | CC | 100 | 66 | 0.22 |
| | | | T | 140 | 71 | | TC | 98 | 59 | |
| | | | | | | | TT | 21 | 6 | |
| 1 | rs649614 | 180051849 | A | 252 | 154 | 0.63 | AA | 67 | 45 | 0.68 |
| | | | G | 194 | 110 | | AG | 118 | 64 | |
| | | | | | | | GG | 38 | 23 | |
| 1 | rs2811558 | 180062070 | G | 240 | 143 | 0.99 | GG | 59 | 39 | 0.56 |
| | | | T | 206 | 123 | | TG | 122 | 65 | |
| | | | | | | | TT | 42 | 29 | |
| 1 | rs2225932 | 180084222 | C | 388 | 224 | 0.30 | CC | 169 | 95 | 0.57 |
| | | | T | 58 | 42 | | TC | 50 | 34 | |
| | | | | | | | TT | 4 | 4 | |
| 1 | rs2788060 | 180094303 | C | 244 | 145 | 0.97 | CC | 62 | 41 | 0.52 |
| | | | T | 198 | 117 | | TC | 120 | 63 | |
| | | | | | | | TT | 39 | 27 | |
| 1 | rs3120798 | 180105322 | C | 306 | 189 | 0.72 | CC | 103 | 64 | 0.88 |
| | | | T | 136 | 79 | | CT | 100 | 61 | |
| | | | | | | | TT | 18 | 9 | |
| 1 | rs4652800 | 180115300 | C | 194 | 113 | 0.71 | CC | 38 | 26 | 0.38 |
| | | | T | 238 | 147 | | TC | 118 | 61 | |
| | | | | | | | TT | 60 | 43 | |
| 1 | rs2811551 | 180129246 | A | 301 | 183 | 0.58 | AA | 102 | 62 | 0.70 |
| | | | C | 139 | 77 | | AC | 97 | 59 | |
| | | | | | | | CC | 21 | 9 | |
| 1 | rs2275675 | 180171140 | A | 284 | 167 | 0.81 | AA | 88 | 46 | 0.32 |
| | | | G | 162 | 99 | | AG | 108 | 75 | |
| | | | | | | | GG | 27 | 12 | |
| 1 | rs2702176 | 180181558 | A | 45 | 26 | 0.95 | AA | 2 | 2 | 0.83 |
| | | | G | 397 | 226 | | GA | 41 | 22 | |
| | | | | | | | GG | 178 | 102 | |
| 1 | rs2702200 | 180208888 | C | 193 | 110 | 0.74 | CC | 48 | 20 | 0.15 |
| | | | T | 243 | 146 | | TC | 97 | 70 | |
| | | | | | | | TT | 73 | 38 | |
| 1 | rs789169 | 180218516 | A | 396 | 242 | 0.90 | AA | 178 | 111 | 0.45 |
| | | | G | 44 | 26 | | AG | 40 | 20 | |
| | | | | | | | GG | 2 | 3 | |
| 1 | rs2877978 | 180228315 | A | 287 | 170 | 0.77 | AA | 99 | 54 | 0.21 |
| | | | G | 149 | 84 | | GA | 89 | 62 | |
| | | | | | | | GG | 30 | 11 | |
| 1 | rs2298083 | 180247085 | A | 53 | 39 | 0.23 | AA | 2 | 1 | 0.38 |
| | | | G | 387 | 217 | | GA | 49 | 37 | |
| | | | | | | | GG | 169 | 90 | |
| 1 | rs2702182 | 180248097 | C | 284 | 165 | 0.85 | CC | 90 | 47 | 0.36 |
| | | | T | 162 | 97 | | TC | 104 | 71 | |
| | | | | | | | TT | 29 | 13 | |
| 1 | rs699245 | 180252940 | C | 229 | 124 | 0.25 | CC | 62 | 24 | 0.08 |
| | | | T | 213 | 138 | | CT | 105 | 76 | |
| | | | | | | | TT | 54 | 31 | |
| 1 | rs789181 | 180257995 | A | 397 | 236 | 0.91 | AA | 178 | 108 | 0.44 |
| | | | G | 45 | 26 | | GA | 41 | 20 | |
| | | | | | | | GG | 2 | 3 | |
| 1 | rs2296164 | 180266592 | C | 236 | 125 | 0.25 | CC | 61 | 25 | 0.23 |
| | | | T | 210 | 133 | | CT | 114 | 75 | |
| | | | | | | | TT | 48 | 29 | |
| 1 | rs2274064 | 180274044 | A | 234 | 128 | 0.32 | AA | 61 | 25 | 0.16 |
| | | | G | 216 | 138 | | GA | 112 | 78 | |
| | | | | | | | GG | 52 | 30 | |
| 1 | rs699242 | 180278130 | C | 83 | 41 | 0.29 | CC | 10 | 4 | 0.59 |
| | | | T | 359 | 221 | | CT | 63 | 33 | |
| | | | | | | | TT | 148 | 94 | |
| 1 | rs4987079 | 180291853 | A | 40 | 31 | 0.26 | AA | 0 | 1 | 0.29 |
| | | | G | 404 | 235 | | AG | 40 | 29 | |
| | | | | | | | GG | 182 | 103 | |
| 1 | rs2333685 | 180297646 | C | 164 | 96 | 0.65 | CC | 32 | 19 | 0.86 |
| | | | T | 254 | 160 | | CT | 100 | 58 | |
| | | | | | | | TT | 77 | 51 | |
| 1 | rs11755 | 180327793 | C | 211 | 125 | 0.80 | CC | 52 | 32 | 0.85 |
| | | | T | 219 | 135 | | CT | 107 | 61 | |
| | | | | | | | TT | 56 | 37 | |
| 1 | rs2767305 | 180336562 | A | 227 | 132 | 0.70 | AA | 60 | 40 | 0.46 |
| | | | G | 205 | 112 | | AG | 107 | 52 | |
| | | | | | | | GG | 49 | 30 | |
| 1 | rs1174656 | 180348344 | A | 147 | 85 | 0.71 | AA | 31 | 18 | 0.92 |
| | | | G | 281 | 173 | | GA | 85 | 49 | |
| | | | | | | | GG | 98 | 62 | |
| 1 | rs1174657 | 180348583 | C | 169 | 94 | 0.51 | CC | 36 | 20 | 0.78 |
| | | | T | 275 | 170 | | CT | 97 | 54 | |
| | | | | | | | TT | 89 | 58 | |
| 1 | rs1174658 | 180348762 | A | 268 | 161 | 0.76 | AA | 80 | 50 | 0.91 |
| | | | G | 180 | 103 | | AG | 108 | 61 | |
| | | | | | | | GG | 36 | 21 | |
| 1 | rs1174679 | 180378011 | C | 163 | 93 | 0.67 | CC | 29 | 19 | 0.57 |
| | | | T | 283 | 173 | | TC | 105 | 55 | |
| | | | | | | | TT | 89 | 59 | |
| 1 | rs4609393 | 180420923 | C | 199 | 127 | 0.50 | CC | 44 | 32 | 0.66 |
| | | | T | 225 | 129 | | TC | 111 | 63 | |
| | | | | | | | TT | 57 | 33 | |
| 1 | rs1073848 | 180430171 | A | 232 | 133 | 0.61 | AA | 59 | 35 | 0.69 |
| | | | G | 129 | 129 | | GA | 114 | 63 | |
| | | | | | | | GG | 47 | 33 | |
| 1 | rs6658466 | 180441005 | C | 237 | 136 | 0.65 | CC | 60 | 35 | 0.79 |
| | | | T | 211 | 130 | | TC | 117 | 66 | |
| | | | | | | | TT | 47 | 32 | |
| 1 | rs6684415 | 180450187 | A | 271 | 171 | 0.86 | AA | 86 | 58 | 0.64 |
| | | | T | 155 | 95 | | TA | 99 | 55 | |
| | | | | | | | TT | 28 | 20 | |
| 1 | rs6679538 | 180468213 | C | 280 | 171 | 0.74 | CC | 87 | 58 | 0.50 |
| | | | T | 164 | 95 | | TC | 106 | 55 | |
| | | | | | | | TT | 29 | 20 | |
| 1 | rs6676389 | 180476550 | A | 253 | 156 | 0.51 | AA | 72 | 48 | 0.72 |
| | | | G | 191 | 106 | | AG | 109 | 60 | |
| | | | | | | | GG | 41 | 23 | |
| 1 | rs1952234 | 180486363 | A | 162 | 92 | 0.78 | AA | 30 | 18 | 0.87 |
| | | | G | 276 | 164 | | AG | 102 | 56 | |
| | | | | | | | GG | 87 | 54 | |
| 1 | rs3814326 | 180504731 | C | 144 | 83 | 0.73 | CC | 25 | 13 | 0.91 |
| | | | T | 300 | 183 | | CT | 94 | 57 | |
| | | | | | | | TT | 103 | 63 | |
| 1 | rs2500111 | 180535764 | C | 363 | 212 | 0.26 | CC | 153 | 88 | 0.4187 |
| | | | T | 71 | 52 | | TC | 57 | 36 | |
| | | | | | | | TT | 7 | 8 | |
| 1 | rs6424914 | 180545515 | G | 409 | 251 | 0.44 | GG | 190 | 117 | 0.3967 |
| | | | T | 35 | 17 | | TG | 29 | 17 | |
| | | | | | | | TT | 3 | 0 | |
| 1 | rs4651156 | 180568689 | A | 129 | 65 | 0.18 | AA | 18 | 8 | 0.3952 |
| | | | G | 299 | 191 | | AG | 93 | 49 | |
| | | | | | | | GG | 103 | 71 | |
| 1 | rs4079923 | 180578941 | A | 188 | 108 | 0.89 | AA | 38 | 26 | 0.4158 |
| | | | T | 228 | 134 | | TA | 112 | 56 | |
| | | | | | | | TT | 58 | 39 | |
| 1 | rs3814330 | 180598742 | C | 319 | 184 | 0.81 | CC | 115 | 69 | 0.4336 |
| | | | T | 113 | 68 | | TC | 89 | 46 | |
| | | | | | | | TT | 12 | 11 | |
| 1 | rs6658545 | 180619259 | A | 344 | 205 | 0.93 | AA | 130 | 82 | 0.1930 |
| | | | G | 104 | 63 | | AG | 84 | 41 | |
| | | | | | | | GG | 10 | 11 | |
| 1 | rs4328036 | 180626835 | A | 244 | 138 | 0.33 | AA | 62 | 39 | 0.0855 |
| | | | G | 182 | 120 | | AG | 120 | 60 | |
| | | | | | | | GG | 31 | 30 | |
| 1 | rs4650 | 180628618 | C | 229 | 136 | 0.76 | CC | 63 | 42 | 0.2737 |
| | | | T | 173 | 108 | | CT | 103 | 52 | |
| | | | | | | | TT | 35 | 28 | |
| 1 | rs1053093 | 180636730 | A | 103 | 64 | 0.90 | AA | 7 | 11 | 0.0436 |
| | | | G | 333 | 202 | | AG | 89 | 42 | |
| | | | | | | | GG | 122 | 80 | |
| 1 | rs3748552 | 180638582 | A | 187 | 113 | 0.60 | AA | 37 | 28 | 0.3862 |
| | | | G | 243 | 135 | | AG | 113 | 57 | |
| | | | | | | | GG | 65 | 39 | |
| 1 | rs734657 | 180643976 | G | 309 | 193 | 0.41 | GG | 108 | 68 | 0.5081 |
| | | | T | 131 | 71 | | TG | 93 | 57 | |
| | | | | | | | TT | 19 | 7 | |
| 1 | rs2986551 | 180654480 | C | 111 | 69 | 0.64 | CC | 12 | 13 | 0.1926 |
| | | | T | 327 | 187 | | TC | 87 | 43 | |
| | | | | | | | TT | 120 | 72 | |
| 1 | rs1887277 | 180664116 | A | 305 | 171 | 0.27 | AA | 108 | 54 | 0.3309 |
| | | | C | 135 | 91 | | AC | 89 | 63 | |
| | | | | | | | CC | 23 | 14 | |
| 1 | rs2296714 | 180674607 | A | 333 | 183 | 0.43 | AA | 126 | 64 | 0.4381 |
| | | | G | 109 | 69 | | GA | 81 | 55 | |
| | | | | | | | GG | 14 | 7 | |
| 1 | rs6424920 | 180704056 | C | 95 | 59 | 0.80 | CC | 16 | 7 | 0.4753 |
| | | | T | 333 | 197 | | TC | 63 | 45 | |
| | | | | | | | TT | 135 | 76 | |
| 1 | rs765225 | 180747364 | C | 382 | 236 | 0.26 | CC | 170 | 107 | 0.46 |
| | | | G | 52 | 24 | | GC | 42 | 22 | |
| | | | | | | | GG | 5 | 1 | |
| 1 | rs1046934 | 180755186 | A | 290 | 184 | 0.47 | AA | 97 | 67 | 0.48 |
| | | | C | 146 | 82 | | AC | 96 | 50 | |
| | | | | | | | CC | 25 | 16 | |
| 1 | rs1952256 | 180766773 | C | 140 | 73 | 0.26 | CC | 23 | 13 | 0.37 |
| | | | T | 286 | 181 | | TC | 94 | 47 | |
| | | | | | | | TT | 96 | 67 | |
| 1 | rs1409811 | 180779768 | C | 95 | 55 | 0.88 | CC | 8 | 4 | 0.96 |
| | | | T | 351 | 209 | | CT | 79 | 47 | |
| | | | | | | | TT | 136 | 81 | |
| 1 | rs6657374 | 180784174 | A | 120 | 84 | 0.16 | AA | 17 | 12 | 0.31 |
| | | | C | 326 | 180 | | AC | 86 | 60 | |
| | | | | | | | CC | 120 | 60 | |
| 2 | rs9425343 | 181395194 | A | 271 | 163 | 0.99 | AA | 87 | 52 | 0.99 |
| | | | C | 165 | 99 | | AC | 97 | 59 | |
| | | | | | | | CC | 34 | 20 | |
| 2 | rs10489579 | 181875378 | A | 290 | 166 | 0.34 | AA | 93 | 52 | 0.54 |
| | | | G | 150 | 100 | | GA | 104 | 62 | |
| | | | | | | | GG | 23 | 19 | |
| 2 | rs6698109 | 181903526 | A | 289 | 171 | 0.99 | AA | 89 | 55 | 0.79 |
| | | | G | 161 | 95 | | AG | 111 | 61 | |
| | | | | | | | GG | 25 | 17 | |
| 2 | rs12041704 | 181972131 | A | 241 | 155 | 0.25 | AA | 65 | 47 | 0.46 |
| | | | G | 203 | 109 | | AG | 111 | 61 | |
| | | | | | | | GG | 46 | 24 | |
| 3** | rs994970 | 182429082 | A | 120 | 71 | 0.88 | AA | 19 | 8 | 0.56 |
| | | | G | 328 | 199 | | GA | 82 | 55 | |
| | | | | | | | GG | 123 | 72 | |
| 3** | rs7544533 | 182498183 | C | 311 | 194 | 0.45 | CC | 112 | 70 | 0.59 |
| | | | T | 135 | 74 | | TC | 87 | 54 | |
| | | | | | | | TT | 24 | 10 | |
| 3** | rs1951517 | 182545155 | A | 142 | 79 | 0.44 | AA | 24 | 12 | 0.75 |
| | | | G | 302 | 191 | | AG | 94 | 55 | |
| | | | | | | | GG | 104 | 68 | |
| 3** | rs1407431 | 182558895 | A | 127 | 67 | 0.28 | AA | 21 | 8 | 0.46 |
| | | | G | 305 | 195 | | GA | 85 | 51 | |
| | | | | | | | GG | 110 | 72 | |
| 3** | rs2057383 | 182568154 | C | 138 | 78 | 0.34 | CC | 22 | 11 | 0.63 |
| | | | G | 280 | 186 | | GC | 94 | 56 | |
| | | | | | | | GG | 93 | 65 | |
| 3** | rs1407426 | 182595392 | C | 119 | 72 | 0.99 | CC | 20 | 11 | 0.94 |
| | | | T | 323 | 196 | | TC | 79 | 50 | |
| | | | | | | | TT | 122 | 73 | |
| 3** | rs726777 | 182604480 | A | 120 | 72 | 0.97 | AA | 20 | 11 | 0.95 |
| | | | T | 328 | 198 | | AT | 80 | 50 | |
| | | | | | | | TT | 124 | 74 | |
| 3** | rs7519247 | 182643354 | A | 316 | 188 | 0.73 | AA | 117 | 68 | 0.93 |
| | | | T | 114 | 72 | | AT | 82 | 52 | |
| | | | | | | | TT | 16 | 10 | |
| 3** | rs6688811 | 182673432 | A | 229 | 137 | 0.92 | AA | 63 | 31 | 0.21 |
| | | | G | 219 | 133 | | GA | 103 | 75 | |
| | | | | | | | GG | 58 | 29 | |
| 3** | rs4650689 | 182713710 | A | 202 | 131 | 0.39 | AA | 45 | 29 | 0.55 |
| | | | T | 238 | 135 | | AT | 112 | 73 | |
| | | | | | | | TT | 63 | 31 | |
| 3** | rs743136 | 182724985 | A | 238 | 138 | 0.48 | AA | 63 | 32 | 0.60 |
| | | | G | 204 | 132 | | GA | 112 | 74 | |
| | | | | | | | GG | 46 | 29 | |
| 3** | rs6425013 | 182744486 | A | 242 | 135 | 0.52 | AA | 64 | 32 | 0.70 |
| | | | G | 206 | 127 | | AG | 114 | 71 | |
| | | | | | | | GG | 46 | 28 | |
| 3** | rs7520459 | 182756662 | A | 232 | 136 | 0.70 | AA | 59 | 32 | 0.85 |
| | | | G | 212 | 132 | | AG | 114 | 72 | |
| | | | | | | | GG | 49 | 30 | |
| 3** | rs970578 | 182770124 | A | 224 | 134 | 0.74 | AA | 59 | 29 | 0.32 |
| | | | G | 216 | 136 | | AG | 106 | 76 | |
| | | | | | | | GG | 55 | 30 | |
| 3** | rs6425016 | 182786834 | A | 226 | 134 | 0.73 | AA | 58 | 29 | 0.44 |
| | | | G | 198 | 124 | | GA | 110 | 76 | |
| | | | | | | | GG | 44 | 24 | |
| 3** | rs1923460 | 182800254 | A | 223 | 134 | 0.91 | AA | 57 | 30 | 0.46 |
| | | | G | 227 | 134 | | GA | 109 | 74 | |
| | | | | | | | GG | 59 | 30 | |
| 3** | rs7552781 | 182807177 | C | 220 | 137 | 0.79 | CC | 56 | 30 | 0.31 |
| | | | T | 226 | 135 | | CT | 108 | 77 | |
| | | | | | | | TT | 59 | 29 | |
| 3** | rs720972 | 182817554 | A | 223 | 134 | 0.86 | AA | 58 | 29 | 0.33 |
| | | | C | 217 | 134 | | CA | 107 | 76 | |
| | | | | | | | CC | 55 | 29 | |
| 3** | rs6659783 | 182827158 | A | 295 | 172 | 0.48 | AA | 99 | 56 | 0.78 |
| | | | T | 153 | 100 | | TA | 97 | 60 | |
| | | | | | | | TT | 28 | 20 | |
| 3** | rs721153 | 182834203 | G | 142 | 97 | 0.27 | GG | 27 | 20 | 0.56 |
| | | | T | 300 | 171 | | GT | 88 | 57 | |
| | | | | | | | TT | 106 | 57 | |
| 3** | rs1322374 | 182845982 | A | 338 | 194 | 0.27 | AA | 130 | 75 | 0.26 |
| | | | G | 106 | 74 | | GA | 78 | 44 | |
| | | | | | | | GG | 14 | 15 | |
| 3** | rs2383457 | 182868229 | C | 153 | 90 | 0.83 | CC | 31 | 11 | 0.10 |
| | | | T | 289 | 176 | | CT | 91 | 68 | |
| | | | | | | | TT | 99 | 54 | |
| 3** | rs651967 | 182896556 | C | 99 | 55 | 0.56 | CC | 15 | 7 | 0.82 |
| | | | T | 347 | 215 | | CT | 69 | 41 | |
| | | | | | | | TT | 139 | 87 | |
| 3 | rs857591 | 182989175 | A | 177 | 105 | 0.87 | AA | 34 | 20 | 0.9845 |
| | | | C | 261 | 159 | | CA | 109 | 65 | |
| | | | | | | | CC | 76 | 47 | |
| 3 | rs1293988 | 182997950 | A | 187 | 114 | 0.59 | AA | 41 | 26 | 0.8699 |
| | | | G | 257 | 144 | | AG | 105 | 62 | |
| | | | | | | | GG | 76 | 41 | |
| 3 | rs2273779 | 183005651 | A | 127 | 80 | 0.85 | AA | 22 | 12 | 0.7799 |
| | | | G | 295 | 180 | | GA | 83 | 56 | |
| | | | | | | | GG | 106 | 62 | |
| 3 | rs7756 | 183013353 | G | 286 | 170 | 0.51 | GG | 93 | 54 | 0.4369 |
| | | | T | 154 | 82 | | TG | 100 | 62 | |
| | | | | | | | TT | 27 | 10 | |
| 3 | rs6657726 | 183018944 | C | 291 | 173 | 1.0 | CC | 97 | 55 | 0.7196 |
| | | | T | 153 | 91 | | CT | 97 | 63 | |
| | | | | | | | TT | 28 | 14 | |
| 3 | rs3766708 | 183027617 | A | 153 | 93 | 0.79 | AA | 27 | 14 | 0.6588 |
| | | | G | 287 | 167 | | AG | 99 | 65 | |
| | | | | | | | GG | 94 | 51 | |
| 3 | rs3817586 | 183035955 | C | 389 | 237 | 0.86 | CC | 175 | 106 | 0.8596 |
| | | | T | 53 | 31 | | CT | 39 | 25 | |
| | | | | | | | TT | 7 | 3 | |
| 3 | rs2272412 | 183046606 | C | 375 | 205 | 0.10 | CC | 163 | 84 | 0.2705 |
| | | | T | 61 | 47 | | CT | 49 | 37 | |
| | | | | | | | TT | 6 | 5 | |
| 3 | rs3753565 | 183048145 | A | 67 | 47 | 0.35 | AA | 7 | 5 | 0.6362 |
| | | | G | 383 | 221 | | GA | 53 | 37 | |
| | | | | | | | GG | 165 | 92 | |
| 3 | rs2178215 | 183055256 | A | 255 | 152 | 0.70 | AA | 81 | 42 | 0.0892 |
| | | | C | 175 | 98 | | AC | 93 | 68 | |
| | | | | | | | CC | 41 | 15 | |
| 3 | rs2037959 | 183067550 | C | 136 | 83 | 0.84 | CC | 22 | 13 | 0.9555 |
| | | | T | 310 | 183 | | CT | 92 | 57 | |
| | | | | | | | TT | 109 | 63 | |
| 3 | rs3131550 | 183085106 | C | 145 | 91 | 0.82 | CC | 25 | 13 | 0.6263 |
| | | | G | 283 | 171 | | GC | 95 | 65 | |
| | | | | | | | GG | 94 | 53 | |
| 3 | rs12084264 | 183094776 | C | 311 | 184 | 0.99 | CC | 108 | 64 | 0.9997 |
| | | | G | 139 | 82 | | GC | 95 | 56 | |
| | | | | | | | GG | 22 | 13 | |
| 3 | rs7539128 | 183116764 | G | 303 | 179 | 0.90 | GG | 108 | 62 | 0.8900 |
| | | | T | 131 | 79 | | TG | 87 | 55 | |
| | | | | | | | TT | 22 | 12 | |
| 3 | rs8824 | 183121960 | A | 180 | 101 | 0.49 | AA | 44 | 16 | 0.0762 |
| | | | C | 254 | 159 | | CA | 92 | 69 | |
| | | | | | | | CC | 81 | 45 | |
| 3 | rs1929092 | 183128165 | A | 123 | 60 | 0.16 | AA | 24 | 10 | 0.42 |
| | | | G | 311 | 196 | | AG | 75 | 40 | |
| | | | | | | | GG | 118 | 78 | |
| 3 | rs4567244 | 183135305 | A | 323 | 202 | 0.35 | AA | 122 | 79 | 0.67 |
| | | | G | 121 | 64 | | GA | 79 | 44 | |
| | | | | | | | GG | 21 | 10 | |
| 3 | rs6675505 | 183138356 | C | 321 | 204 | 0.17 | CC | 121 | 80 | 0.40 |
| | | | T | 125 | 62 | | CT | 79 | 44 | |
| | | | | | | | TT | 23 | 9 | |
| 3 | rs6677914 | 183154998 | C | 389 | 233 | 0.77 | CC | 170 | 104 | 0.73 |
| | | | T | 59 | 33 | | TC | 49 | 25 | |
| | | | | | | | TT | 5 | 4 | |
| 3 | rs1799957 | 183163002 | G | 123 | 63 | 0.25 | GG | 22 | 9 | 0.52 |
| | | | T | 323 | 203 | | TG | 79 | 45 | |
| | | | | | | | TT | 122 | 79 | |
| 3 | rs5275 | 183374715 | C | 147 | 89 | 0.85 | CC | 28 | 16 | 0.91 |
| | | | T | 281 | 165 | | TC | 91 | 57 | |
| | | | | | | | TT | 95 | 54 | |
| 3 | rs3820185 | 183540000 | A | 145 | 80 | 0.35 | AA | 27 | 11 | 0.47 |
| | | | C | 279 | 180 | | CA | 91 | 58 | |
| | | | | | | | CC | 94 | 61 | |
| 3 | rs2076075 | 183554941 | A | 50 | 37 | 0.30 | AA | 3 | 0 | 0.10 |
| | | | G | 394 | 229 | | GA | 44 | 37 | |
| | | | | | | | GG | 175 | 96 | |
| 3 | rs7554339 | 183564551 | A | 350 | 228 | 0.022 | AA | 142 | 98 | 0.069 |
| | | | T | 90 | 36 | | TA | 66 | 32 | |
| | | | | | | | TT | 12 | 2 | |
| 3 | rs1980444 | 183582617 | C | 358 | 215 | 0.76 | CC | 146 | 92 | 0.096 |
| | | | T | 80 | 51 | | TC | 66 | 31 | |
| | | | | | | | TT | 7 | 10 | |
| 3 | rs7519192 | 183594076 | A | 314 | 180 | 0.57 | AA | 114 | 61 | 0.59 |
| | | | G | 130 | 82 | | GA | 86 | 58 | |
| | | | | | | | GG | 22 | 12 | |
| 3 | rs1569480 | 183606118 | A | 226 | 128 | 0.66 | AA | 62 | 36 | 0.78 |
| | | | G | 204 | 124 | | AG | 102 | 56 | |
| | | | | | | | GG | 51 | 34 | |
| 3 | rs6662687 | 183611548 | A | 217 | 131 | 0.82 | AA | 55 | 37 | 0.65 |
| | | | G | 225 | 131 | | AG | 107 | 57 | |
| | | | | | | | GG | 59 | 37 | |
| 3 | rs4336803 | 183626059 | G | 331 | 204 | 0.81 | GG | 130 | 83 | 0.64 |
| | | | T | 85 | 50 | | TG | 71 | 38 | |
| | | | | | | | TT | 7 | 6 | |
| 3 | rs1160719 | 183632124 | A | 85 | 49 | 0.83 | AA | 11 | 4 | 0.63 |
| | | | G | 357 | 215 | | GA | 63 | 41 | |
| | | | | | | | GG | 147 | 87 | |
| 3 | rs4433346 | 183633970 | A | 359 | 218 | 0.65 | AA | 147 | 89 | 0.66 |
| | | | G | 83 | 46 | | GA | 65 | 40 | |
| | | | | | | | GG | 9 | 3 | |
| 3 | rs6697145 | 183642248 | C | 230 | 137 | 0.95 | CC | 62 | 42 | 0.39 |
| | | | T | 212 | 125 | | CT | 106 | 53 | |
| | | | | | | | TT | 53 | 36 | |
| 3 | rs4651343 | 183649644 | A | 130 | 81 | 0.71 | AA | 23 | 12 | 0.62 |
| | | | C | 316 | 185 | | AC | 84 | 57 | |
| | | | | | | | CC | 116 | 64 | |
| 3 | rs1555204 | 183688872 | C | 87 | 57 | 0.51 | CC | 9 | 7 | 0.79 |
| | | | T | 355 | 205 | | CT | 69 | 43 | |
| | | | | | | | TT | 143 | 81 | |
| 3 | rs2144000 | 183698899 | A | 183 | 126 | 0.11 | AA | 39 | 29 | 0.26 |
| | | | G | 261 | 140 | | GA | 105 | 68 | |
| | | | | | | | GG | 78 | 36 | |
| 3 | rs6657726 | 183018944 | C | 291 | 173 | 1.0 | CC | 97 | 55 | 0.72 |
| | | | T | 153 | 91 | | CT | 97 | 63 | |
| | | | | | | | TT | 28 | 14 | |
| 3 | rs3766708 | 183027617 | A | 153 | 93 | 0.79 | AA | 27 | 14 | 0.66 |
| | | | G | 287 | 167 | | AG | 99 | 65 | |
| | | | | | | | GG | 94 | 51 | |
| 3 | rs3817586 | 183035955 | C | 389 | 237 | 0.86 | CC | 175 | 106 | 0.86 |
| | | | T | 53 | 31 | | CT | 39 | 25 | |
| | | | | | | | TT | 7 | 3 | |
| 4 | rs6659885 | 186925883 | C | 248 | 145 | 0.54 | CC | 78 | 44 | 0.84 |
| | | | G | 172 | 111 | | CG | 92 | 57 | |
| | | | | | | | GG | 40 | 27 | |
| 4 | rs7513756 | 186942826 | C | 184 | 115 | 0.60 | CC | 43 | 28 | 0.88 |
| | | | T | 262 | 151 | | TC | 98 | 59 | |
| | | | | | | | TT | 82 | 46 | |
| 5 | rs10921310 | 189806308 | A | 322 | 208 | 0.090 | AA | 114 | 83 | 0.11 |
| | | | T | 126 | 60 | | TA | 94 | 42 | |
| | | | | | | | TT | 16 | 9 | |
| 6 | rs12138336 | 193802178 | C | 408 | 239 | 0.80 | CC | 191 | 110 | 0.42 |
| | | | G | 30 | 19 | | CG | 26 | 19 | |
| | | | | | | | GG | 2 | 0 | |
| 6 | rs1998712 | 192919322 | A | 360 | 220 | 0.51 | AA | 146 | 92 | 0.77 |
| | | | G | 86 | 46 | | GA | 68 | 36 | |
| | | | | | | | GG | 9 | 5 | |
| 6 | rs1925332 | 192945224 | C | 334 | 210 | 0.39 | CC | 137 | 90 | 0.58 |
| | | | G | 80 | 42 | | GC | 60 | 30 | |
| | | | | | | | GG | 10 | 6 | |
| 6 | rs2477361 | 192955995 | A | 356 | 220 | 0.34 | AA | 142 | 93 | 0.41 |
| | | | G | 90 | 46 | | AG | 72 | 34 | |
| | | | | | | | GG | 9 | 6 | |
| 6 | rs660711 | 192967683 | A | 346 | 210 | 0.40 | AA | 139 | 89 | 0.46 |
| | | | G | 86 | 44 | | AG | 68 | 32 | |
| | | | | | | | GG | 9 | 6 | |
| 6 | rs1325299 | 192978579 | C | 396 | 233 | 0.96 | CC | 178 | 107 | 0.21 |
| | | | T | 42 | 25 | | CT | 40 | 19 | |
| | | | | | | | TT | 1 | 3 | |
| 6 | rs1924757 | 192991686 | A | 91 | 44 | 0.24 | AA | 9 | 6 | 0.26 |
| | | | G | 353 | 216 | | GA | 73 | 32 | |
| | | | | | | | GG | 140 | 92 | |
| 6 | rs2181948 | 193013459 | A | 196 | 114 | 0.78 | AA | 46 | 26 | 0.97 |
| | | | G | 250 | 152 | | AG | 104 | 62 | |
| | | | | | | | GG | 73 | 45 | |
| 6 | rs4379659 | 193048418 | C | 357 | 222 | 0.36 | CC | 143 | 94 | 0.43 |
| | | | T | 89 | 46 | | CT | 71 | 34 | |
| | | | | | | | TT | 9 | 6 | |
| 6 | rs7552975 | 193071312 | A | 87 | 44 | 0.28 | AA | 9 | 6 | 0.33 |
| | | | G | 349 | 220 | | AG | 69 | 32 | |
| | | | | | | | GG | 140 | 94 | |
| 6 | rs1416962 | 193186285 | A | 288 | 148 | 0.0089 | AA | 100 | 36 | 0.0018 |
| | | | G | 140 | 110 | | GA | 88 | 76 | |
| | | | | | | | GG | 26 | 17 | |
| 6 | rs3753394 | 193352574 | C | 334 | 192 | 0.49 | CC | 122 | 70 | 0.56 |
| | | | T | 114 | 74 | | TC | 90 | 52 | |
| | | | | | | | TT | 12 | 11 | |
| 6 | rs800292 | 193373890 | C | 368 | 196 | 0.0009 | CC | 160 | 73 | 0.0022 |
| | | | T | 60 | 62 | | TC | 48 | 50 | |
| | | | | | | | TT | 6 | 6 | |
| 6 | rs572515 | 193377918 | A | 248 | 90 | 3.6E−08 | AA | 71 | 15 | 6.8E−07 |
| | | | G | 202 | 176 | | AG | 106 | 60 | |
| | | | | | | | GG | 48 | 58 | |
| 6 | rs1329423 | 193378044 | C | 106 | 68 | 0.51 | CC | 13 | 11 | 0.65 |
| | | | T | 344 | 196 | | TC | 80 | 46 | |
| | | | | | | | TT | 132 | 75 | |
| 6 | rs3766404 | 193383489 | C | 37 | 49 | 6.1E−05 | CC | 3 | 4 | 0.0002 |
| | | | T | 409 | 217 | | CT | 31 | 41 | |
| | | | | | | | TT | 189 | 88 | |
| 6 | rs1061147 | 193385981 | A | 249 | 92 | 5E−08 | AA | 72 | 15 | 8.7E−07 |
| | | | C | 201 | 176 | | CA | 105 | 62 | |
| | | | | | | | CC | 48 | 57 | |
| 6 | rs1061170 | 193390894 | C | 249 | 89 | 3.7E−08 | CC | 72 | 15 | 7.7E−07 |
| | | | T | 201 | 173 | | TC | 105 | 59 | |
| | | | | | | | TT | 48 | 57 | |
| 6 | rs10922094 | 193393162 | C | 198 | 176 | 9.6E−09 | CC | 48 | 58 | 2.8E−07 |
| | | | G | 252 | 90 | | GC | 102 | 60 | |
| | | | | | | | GG | 75 | 15 | |
| 6 | rs1292471 | 193399085 | A | 201 | 175 | 8.4E−08 | AA | 48 | 58 | 1.6E−06 |
| | | | T | 245 | 91 | | AT | 105 | 59 | |
| | | | | | | | TT | 70 | 16 | |
| 6 | rs2860102 | 193399976 | A | 249 | 92 | 5E−08 | AA | 72 | 16 | 1E−06 |
| | | | T | 201 | 176 | | TA | 105 | 60 | |
| | | | | | | | TT | 48 | 58 | |
| 6 | rs2019724 | 193406574 | C | 183 | 164 | 5.6E−08 | CC | 39 | 51 | 9.7E−07 |
| | | | T | 267 | 102 | | TC | 105 | 62 | |
| | | | | | | | TT | 81 | 20 | |
| 6 | rs10465586 | 193418986 | A | 342 | 150 | 2.3E−08 | AA | 136 | 43 | 5.7E−07 |
| | | | T | 108 | 118 | | TA | 70 | 64 | |
| | | | | | | | TT | 19 | 27 | |
| 6 | rs3753396 | 193427399 | A | 375 | 221 | 0.93 | AA | 153 | 92 | 0.48 |
| | | | G | 75 | 45 | | AG | 69 | 37 | |
| | | | | | | | GG | 3 | 4 | |
| 6 | rs403846 | 193428394 | A | 261 | 101 | 8.9E−08 | AA | 79 | 20 | 1.5E−06 |
| | | | G | 181 | 163 | | AG | 103 | 61 | |
| | | | | | | | GG | 39 | 51 | |
| 6 | rs1065489 | 193441431 | G | 372 | 218 | 0.98 | GG | 151 | 90 | 0.69 |
| | | | T | 78 | 46 | | TG | 70 | 38 | |
| | | | | | | | TT | 4 | 4 | |
| 6 | rs4230 | 193532699 | G | 324 | 175 | 0.066 | GG | 121 | 56 | 0.087 |
| | | | T | 124 | 91 | | TG | 82 | 63 | |
| | | | | | | | TT | 21 | 14 | |
| 6 | rs1805287 | 193542472 | C | 177 | 164 | 3.9E−09 | CC | 44 | 55 | 2E−07 |
| | | | T | 235 | 82 | | CT | 89 | 54 | |
| | | | | | | | TT | 73 | 14 | |
| 6 | rs6428375 | 193604459 | C | 341 | 181 | 0.012 | CC | 135 | 65 | 0.057 |
| | | | T | 101 | 83 | | CT | 71 | 51 | |
| | | | | | | | TT | 15 | 16 | |
| 6 | rs7417769 | 193608115 | A | 102 | 83 | 0.0075 | AA | 15 | 16 | 0.0389 |
| | | | G | 346 | 177 | | GA | 72 | 51 | |
| | | | | | | | GG | 137 | 63 | |
| 6 | rs1853883 | 193613257 | C | 267 | 113 | 5E−06 | CC | 86 | 27 | 9.7E−05 |
| | | | G | 177 | 153 | | CG | 95 | 59 | |
| | | | | | | | GG | 41 | 47 | |
| 6 | rs379370 | 193615850 | C | 327 | 160 | 0.0004 | CC | 117 | 49 | 0.0012 |
| | | | T | 121 | 106 | | TC | 93 | 62 | |
| | | | | | | | TT | 14 | 22 | |
| 6 | rs1971579 | 193618838 | A | 338 | 179 | 0.034 | AA | 129 | 62 | 0.054 |
| | | | C | 88 | 69 | | AC | 80 | 55 | |
| | | | | | | | CC | 4 | 7 | |
| 6 | rs3915683 | 193618931 | A | 67 | 42 | 0.80 | AA | 7 | 4 | 0.93 |
| | | | G | 381 | 226 | | GA | 53 | 34 | |
| | | | | | | | GG | 164 | 96 | |
| 6 | rs4915318 | 193628745 | A | 74 | 69 | 0.0024 | AA | 9 | 10 | 0.012 |
| | | | C | 374 | 197 | | AC | 56 | 49 | |
| | | | | | | | CC | 159 | 74 | |
| 6 | rs3790414 | 193651956 | A | 72 | 67 | 0.0025 | AA | 9 | 9 | 0.0096 |
| | | | T | 376 | 197 | | TA | 54 | 49 | |
| | | | | | | | TT | 161 | 74 | |
| 6 | rs7531555 | 193660967 | C | 362 | 194 | 0.0057 | CC | 155 | 74 | 0.023 |
| | | | T | 70 | 64 | | TC | 52 | 46 | |
| | | | | | | | TT | 9 | 9 | |
| 6 | rs6428379 | 193669193 | C | 178 | 149 | 3.8E−05 | CC | 41 | 46 | 0.0005 |
| | | | T | 262 | 115 | | TC | 96 | 57 | |
| | | | | | | | TT | 83 | 29 | |
| 6 | rs10922152 | 193694663 | A | 279 | 137 | 0.0059 | AA | 86 | 36 | 0.0223 |
| | | | T | 171 | 129 | | TA | 107 | 65 | |
| | | | | | | | TT | 32 | 32 | |
| 6 | rs2336595 | 193732133 | A | 24 | 32 | 0.0016 | AA | 2 | 1 | 0.0019 |
| | | | G | 422 | 236 | | AG | 20 | 30 | |
| | | | | | | | GG | 201 | 103 | |
| 6 | rs698859 | 193740022 | A | 198 | 110 | 0.43 | AA | 49 | 20 | 0.2076 |
| | | | G | 248 | 156 | | AG | 100 | 70 | |
| | | | | | | | GG | 74 | 43 | |
| 6 | rs5998 | 193741455 | C | 224 | 126 | 0.44 | CC | 56 | 28 | 0.6709 |
| | | | T | 224 | 142 | | TC | 112 | 70 | |
| | | | | | | | TT | 56 | 36 | |
| 6 | rs1615413 | 193761974 | A | 222 | 141 | 0.43 | AA | 54 | 36 | 0.7197 |
| | | | G | 226 | 127 | | GA | 114 | 69 | |
| | | | | | | | GG | 56 | 29 | |
| 6 | rs6003 | 193762678 | A | 423 | 231 | 0.0029 | AA | 201 | 101 | 0.0038 |
| | | | G | 25 | 31 | | AG | 21 | 29 | |
| | | | | | | | GG | 2 | 1 | |
| 6 | rs6428381 | 193770270 | A | 26 | 33 | 0.0022 | AA | 2 | 1 | 0.0028 |
| | | | C | 418 | 233 | | AC | 22 | 31 | |
| | | | | | | | CC | 198 | 101 | |
| 6 | rs3762271 | 193802099 | A | 198 | 112 | 0.56 | AA | 48 | 20 | 0.2076 |
| | | | C | 252 | 156 | | CA | 102 | 72 | |
| | | | | | | | CC | 75 | 42 | |
| 6 | rs4915327 | 193820650 | C | 402 | 228 | 0.0021 | CC | 191 | 100 | 0.0061 |
| | | | T | 22 | 30 | | TC | 20 | 28 | |
| | | | | | | | TT | 1 | 1 | |
| 6 | rs12116571 | 193820650 | C | 364 | 244 | 0.0002 | CC | 151 | 114 | 0.0006 |
| | | | T | 68 | 16 | | CT | 62 | 16 | |
| | | | | | | | TT | 3 | 0 | |
| 6 | rs10732295 | 193868302 | C | 409 | 233 | 0.002 | CC | 194 | 101 | 0.0023 |
| | | | T | 25 | 33 | | TC | 21 | 31 | |
| | | | | | | | TT | 2 | 1 | |
| 6 | rs10429911 | 193877801 | A | 22 | 28 | 0.0072 | AA | 2 | 1 | 0.0094 |
| | | | C | 388 | 226 | | AC | 18 | 26 | |
| | | | | | | | CC | 185 | 100 | |
| 6 | rs10737688 | 193887712 | C | 228 | 131 | 0.79 | CC | 54 | 32 | 0.856 |
| | | | T | 212 | 127 | | TC | 120 | 67 | |
| | | | | | | | TT | 46 | 30 | |
| 6 | rs10922181 | 193897709 | C | 169 | 102 | 0.93 | CC | 35 | 14 | 0.105 |
| | | | G | 279 | 166 | | CG | 99 | 74 | |
| | | | | | | | GG | 90 | 46 | |
| 6 | rs489839 | 193970506 | C | 341 | 204 | 0.69 | CC | 134 | 79 | 0.5855 |
| | | | G | 101 | 56 | | CG | 73 | 46 | |
| | | | | | | | GG | 14 | 5 | |
| 6 | rs7548481 | 193979471 | A | 274 | 159 | 0.92 | AA | 88 | 47 | 0.43 |
| | | | G | 170 | 97 | | AG | 98 | 65 | |
| | | | | | | | GG | 36 | 16 | |
| 6 | rs7418217 | 194003905 | G | 171 | 103 | 0.98 | GG | 36 | 17 | 0.39 |
| | | | T | 275 | 165 | | TG | 99 | 69 | |
| | | | | | | | TT | 88 | 48 | |
| 6 | rs1764622 | 194013736 | C | 285 | 176 | 0.81 | CC | 94 | 57 | 0.84 |
| | | | T | 155 | 92 | | TC | 97 | 62 | |
| | | | | | | | TT | 29 | 15 | |
| 6 | rs570845 | 194024406 | A | 290 | 176 | 0.86 | AA | 95 | 57 | 0.92 |
| | | | T | 156 | 92 | | AT | 100 | 62 | |
| | | | | | | | TT | 28 | 15 | |
| 6 | rs2786104 | 194044091 | A | 37 | 27 | 0.42 | AA | 2 | 1 | 0.63 |
| | | | T | 405 | 239 | | TA | 33 | 25 | |
| | | | | | | | TT | 186 | 107 | |
| 6 | rs10922213 | 194083923 | C | 301 | 178 | 0.94 | CC | 99 | 57 | 0.92 |
| | | | T | 147 | 88 | | TC | 103 | 64 | |
| | | | | | | | TT | 22 | 12 | |
| 6 | rs10922216 | 194103855 | A | 288 | 171 | 0.76 | AA | 98 | 55 | 0.74 |
| | | | C | 136 | 85 | | AC | 92 | 61 | |
| | | | | | | | CC | 22 | 12 | |
| 6 | rs2821121 | 194113713 | C | 295 | 174 | 0.96 | CC | 96 | 56 | 0.97 |
| | | | T | 147 | 86 | | TC | 103 | 62 | |
| | | | | | | | TT | 22 | 12 | |
| 6 | rs4915545 | 194143686 | C | 242 | 141 | 0.96 | CC | 63 | 33 | 0.59 |
| | | | T | 196 | 115 | | TC | 116 | 75 | |
| | | | | | | | T | 40 | 20 | |
| 6 | rs10922234 | 194167696 | A | 353 | 212 | 0.95 | AA | 139 | 86 | 0.62 |
| | | | G | 91 | 54 | | AG | 75 | 40 | |
| | | | | | | | GG | 8 | 7 | |
| 6 | rs10922237 | 194178359 | G | 237 | 150 | 0.44 | GG | 59 | 38 | 0.64 |
| | | | T | 203 | 114 | | TG | 119 | 74 | |
| | | | | | | | TT | 42 | 20 | |
| 6 | rs10801614 | 194251930 | C | 355 | 216 | 0.56 | CC | 142 | 91 | 0.40 |
| | | | T | 85 | 46 | | TC | 71 | 34 | |
| | | | | | | | TT | 7 | 6 | |
| 6 | rs7516153 | 194273380 | C | 198 | 122 | 0.70 | CC | 41 | 24 | 0.76 |
| | | | T | 238 | 138 | | CT | 116 | 74 | |
| | | | | | | | TT | 61 | 32 | |
| 6 | rs4915555 | 194283171 | C | 342 | 209 | 0.88 | CC | 135 | 86 | 0.43 |
| | | | G | 86 | 51 | | GC | 72 | 37 | |
| | | | | | | | GG | 7 | 7 | |
| 6 | rs6676795 | 194302896 | C | 84 | 50 | 0.88 | CC | 6 | 7 | 0.28 |
| | | | G | 336 | 206 | | CG | 72 | 36 | |
| | | | | | | | GG | 132 | 85 | |
| 6 | rs12116508 | 194312109 | C | 93 | 52 | 0.77 | CC | 7 | 8 | 0.17 |
| | | | T | 345 | 204 | | TC | 79 | 36 | |
| | | | | | | | TT | 133 | 84 | |
| 6 | rs10801620 | 194323063 | A | 205 | 125 | 0.79 | AA | 44 | 25 | 0.76 |
| | | | C | 241 | 141 | | CA | 117 | 75 | |
| | | | | | | | CC | 62 | 33 | |
| 6 | rs12127378 | 194332749 | C | 93 | 52 | 0.70 | CC | 6 | 8 | 0.085 |
| | | | T | 345 | 208 | | TC | 81 | 36 | |
| | | | | | | | TT | 132 | 86 | |
| 6 | rs9427669 | 194351589 | C | 349 | 212 | 0.73 | CC | 134 | 87 | 0.17 |
| | | | T | 95 | 54 | | TC | 31 | 38 | |
| | | | | | | | TT | 7 | 8 | |
| 6 | rs2488389 | 194362798 | C | 331 | 200 | 0.39 | CC | 123 | 77 | 0.67 |
| | | | T | 109 | 56 | | TC | 85 | 46 | |
| | | | | | | | TT | 12 | 5 | |
| 6 | rs2488410 | 194382162 | A | 93 | 50 | 0.66 | AA | 7 | 8 | 0.12 |
| | | | G | 341 | 200 | | AG | 79 | 34 | |
| | | | | | | | GG | 131 | 83 | |
| 6 | rs1891497 | 194391212 | A | 91 | 54 | 0.92 | AA | 6 | 8 | 0.15 |
| | | | G | 347 | 210 | | AG | 79 | 38 | |
| | | | | | | | GG | 134 | 86 | |
| 6 | rs1747815 | 194429760 | A | 70 | 39 | 0.91 | AA | 6 | 6 | 0.40 |
| | | | G | 334 | 191 | | GA | 58 | 27 | |
| | | | | | | | GG | 138 | 82 | |
| 6 | rs7533766 | 194439771 | A | 180 | 116 | 0.33 | AA | 34 | 24 | 0.60 |
| | | | G | 254 | 140 | | GA | 112 | 68 | |
| | | | | | | | GG | 71 | 36 | |
| 6 | rs1998598 | 194459299 | A | 312 | 179 | 0.59 | AA | 110 | 61 | 0.86 |
| | | | G | 132 | 83 | | AG | 92 | 57 | |
| | | | | | | | GG | 20 | 13 | |
| 6 | rs9427953 | 194490552 | C | 337 | 210 | 0.94 | CC | 137 | 88 | 0.55 |
| | | | T | 75 | 46 | | CT | 63 | 34 | |
| | | | | | | | TT | 6 | 6 | |
| 6 | rs10922288 | 194501197 | C | 99 | 52 | 0.44 | CC | 9 | 6 | 0.52 |
| | | | T | 351 | 214 | | TC | 81 | 40 | |
| | | | | | | | TT | 135 | 87 | |
| 6 | rs6704186 | 194525344 | C | 88 | 51 | 0.75 | CC | 5 | 6 | 0.28 |
| | | | T | 332 | 205 | | TC | 78 | 39 | |
| | | | | | | | TT | 127 | 83 | |
| 6 | rs10922324 | 194599062 | A | 352 | 217 | 0.67 | AA | 142 | 88 | 0.85 |
| | | | T | 90 | 51 | | TA | 68 | 41 | |
| | | | | | | | TT | 11 | 5 | |
| 6 | rs10754236 | 194609145 | C | 281 | 155 | 0.18 | CC | 88 | 43 | 0.37 |
| | | | T | 163 | 111 | | TC | 105 | 69 | |
| | | | | | | | TT | 29 | 21 | |
| 6 | rs10494762 | 194619495 | C | 94 | 66 | 0.29 | CC | 9 | 9 | 0.48 |
| | | | G | 346 | 200 | | CG | 76 | 48 | |
| | | | | | | | GG | 135 | 76 | |
| 6 | rs10801633 | 194629153 | C | 241 | 121 | 0.032 | CC | 74 | 32 | 0.13 |
| | | | T | 201 | 141 | | TC | 93 | 57 | |
| | | | | | | | TT | 54 | 42 | |
| 6 | rs6671069 | 194644758 | C | 259 | 142 | 0.24 | CC | 80 | 44 | 0.37 |
| | | | T | 185 | 122 | | TC | 99 | 54 | |
| | | | | | | | TT | 43 | 34 | |

*The number of SNPs successfully genotyped in clusters 1, 2, 3, 4, 5, and 6 were 82, 4, 61, 2, 1, and 86. All SNPs reported in this manuscript are included in this table.
**Two asterisks in the “Cluster” column denote SNPs within the Fibulin 6 locus referred to in the text.

TABLE 5
Genotyping in the replication sample: allele and genotype association between AMD
and 14 polymorphic SNPs in the RCA locus.

| | | | | | | χ²_{1 df} | | | | χ²_{2 df} |
|---|---|---|---|---|---|---|---|---|---|---|
| | | | Allelle Counts | | | Test | Genotype Counts | | | Test |
| Cluster | SNP Name | Location | | AMD | Control | p-value | | AMD | Control | p-value |
| 6 | rs3753394 | 193352574 | C | 240 | 93 | 0.97 | CC | 86 | 33 | 0.99 |
| | | | T | 110 | 43 | | TC | 68 | 27 | |
| | | | | | | | TT | 21 | 8 | |
| 6 | rs800292 | 193373890 | A | 43 | 23 | 0.15 | AA | 1 | 4 | 0.030 |
| | | | G | 309 | 111 | | AG | 41 | 15 | |
| | | | | | | | GG | 134 | 48 | |
| 6 | rs1061170 | 193390894 | C | 185 | 46 | 0.0039 | CC | 52 | 11 | 0.014 |
| | | | T | 155 | 72 | | TC | 81 | 24 | |
| | | | | | | | TT | 37 | 24 | |
| 6 | rs2019724 | 193406574 | C | 138 | 75 | 0.0020 | CC | 29 | 23 | 0.0082 |
| | | | T | 210 | 61 | | TC | 80 | 29 | |
| | | | | | | | TT | 65 | 16 | |
| 6 | rs1065489 | 193441431 | G | 290 | 108 | 0.38 | GG | 123 | 43 | 0.52 |
| | | | T | 60 | 28 | | TG | 44 | 22 | |
| | | | | | | | TT | 8 | 3 | |
| 6 | rs4230 | 193532699 | G | 257 | 89 | 0.099 | GG | 93 | 32 | 0.081 |
| | | | T | 95 | 47 | | TG | 71 | 25 | |
| | | | | | | | TT | 12 | 11 | |
| 6 | rs7417769 | 193608115 | A | 76 | 43 | 0.010 | AA | 10 | 8 | 0.047 |
| | | | G | 274 | 87 | | GA | 56 | 27 | |
| | | | | | | | GG | 109 | 30 | |
| 6 | rs379370 | 193615850 | C | 254 | 82 | 0.026 | CC | 94 | 28 | 0.074 |
| | | | T | 96 | 50 | | TC | 66 | 26 | |
| | | | | | | | TT | 15 | 12 | |
| 6 | rs3915683 | 193618931 | A | 54 | 28 | 0.16 | AA | 7 | 3 | 0.28 |
| | | | G | 298 | 108 | | GA | 40 | 22 | |
| | | | | | | | GG | 129 | 43 | |
| 6 | rs3790414 | 193651956 | A | 57 | 25 | 0.58 | AA | 3 | 4 | 0.20 |
| | | | T | 293 | 111 | | TA | 51 | 17 | |
| | | | | | | | TT | 121 | 47 | |
| 6 | rs10922152 | 193694663 | A | 226 | 71 | 0.015 | AA | 74 | 16 | 0.026 |
| | | | T | 126 | 65 | | TA | 78 | 39 | |
| | | | | | | | TT | 24 | 13 | |
| 6 | rs5998 | 193741455 | C | 160 | 66 | 0.54 | CC | 38 | 14 | 0.46 |
| | | | T | 192 | 70 | | TC | 84 | 38 | |
| | | | | | | | TT | 54 | 16 | |
| 6 | rs6003 | 193762678 | A | 332 | 123 | 0.074 | AA | 157 | 57 | 0.057 |
| | | | G | 18 | 13 | | AG | 18 | 9 | |
| | | | | | | | CC | 0 | 2 | |
| 6 | rs3762271 | 193802099 | A | 173 | 55 | 0.084 | AA | 46 | 11 | 0.22 |
| | | | C | 179 | 81 | | CA | 81 | 33 | |
| | | | | | | | CC | 49 | 24 | |

TABLE 6
SEQUENCES

| Sequence Name | |
|---|---|
| and SEQ ID NO: | Sequence |
| RS1061170-1277F | 5′ CTTTATTTATTTATCATTGTTATGGTCCT |
| (SEQ ID NO:3) | TAGGAAAATGTTATTT 3′ |
| RS1061170-1277R | 5′ GGCAGGCAACGTCTATAGATTTACC 3′ |
| (SEQ ID NO:4) | |
| RS106117077V2 | 5′ TTTCTTCCATGATTTTG 3′ |
| (SEQ ID NO:5) | |
| RS1061170-1277M2 | 5′ TTCTTCCATAATTTTG 3′ |
| (SEQ ID NO:6) | |
| Rs1061170 (short) | 5′ AAAATGGATATAATCAAAATTATGAAGAA |
| (SEQ ID NO:7) | AGTTTGTACAG 3′ |
| Rs1061170 (long) | 5′ CTGGGATCACATTCATTGCACACAAGATG |
| (SEQ ID NO:8) | GATGGTCGCCAGCAGTACCATGCCTCAGAAAA |
| | TGTTATTTTCCTTATTTGGAAAATGGATATAA |
| | TCAAAATTATGGAAGAAAGTTTGTACAGGGTA |
| | AATCTATAGACGTTGCCTGCCATCCTGGCTAC |
| | GCTCTTCCAAAAGCGCAGACCACAGTTACATG |
| | TATGGAGAATGG 3′ |

### REFERENCES

1. R. Klein, B. E. Klein, S. C. Tomany, S. M. Meuer, G. H. Huang, Ophthalmology 109, 1767 (2002).
2. R. Klein, T. Peto, A. Bird, M. R. Vannewkirk, Am. J. Ophthalmol. 137, 486 (2004).
3. G. R. Abecasis et al., Am. J. Hum. Genet. 74, 482 (2004).
4. S. K. lyengar et al., Am. J Hum. Genet. 74, 20 (2004).
5. M. L. Klein et al., Arch. Ophthalmol. 116, 1082 (1998).
6. J. Majewski et al., Am. J. Hum. Genet. 73, 540 (2003).
7. J. H. Schick et al., Am. J. Hum. Genet. 72, 1412 (2003).
8. J. M. Seddon, S. L. Santangelo, K. Book, S. Chong, J. Cote, Am. J. Hum. Genet. 73, 780 (2003).
9. D. E. Weeks et al., Am. J. Hum. Genet. 75, 174 (2004).
10. Materials and methods are described above.
11. D. W. Schultz et al., Hum. Mol. Genet. 12, 3315 (2003).
12. D. J. Schaid, C. M. Rowland, D. E. Tines, R. M. Jacobson, G. A. Poland, Am. J. Hum. Genet. 70, 425 (2002).
13. H. Zhao, R. Pfeiffer, M. H. Gail, Pharmacogenomics 4, 171 (2003).
14. J. Acosta, X. Qin, J. Halperin, Curr. Pharm. Des. 10, 203 (2004).
15. H. J. Muller-Eberhard, R. D. Schreiber, Adv. Immunol. 29, 1 (1980).
16. R. F. Mullins, S. R. Russell, D. H. Anderson, G. S. Hageman, FASEB J. 14, 835 (2000).
17. L. V. Johnson, W. P. Leitner, M. K. Staples, D. H. Anderson, Exp. Eye. Res. 73, 887 (2001).
18. E. Giannakis et al., Eur. J. Immunol. 33, 962 (2003).
19. J. M. Seddon, G. Gensler, R. C. Milton, M. L. Klein, N. Rifai, JAMA 291, 704 (2004).
20. C. Mold, H. Gewurz, T. W. Du Clos, Immunopharmacology 42, 23 (1999).
21. J. M. Seddon, J. Cote, N. Davis, B. Rosner, Arch. Ophthalmol. 121, 785 (2003).
22. J. Esparza-Gordillo et al., Immunogenetics 56, 77 (2004).
23. J. R. Greenfield et al., Circulation 109, 3022 (2004).
24. J. M. Backes, P. A. Howard, P. M. Moriarty, Ann. Pharmacother. 38, 110 (2004).
25. M. H. Wener, P. R. Daum, G. M. McQuillan, J Rheumatol. 27, 2351 (2000).
26. R. F. Mullins, N. Aptsiauri, G. S. Hageman, Eye 15, 390 (2001).
27. M. A. Dragon-Durey et al., J. Am. Soc. Nephrol. 15, 787 (2004).
28. D. S. Friedman et al., Arch. Ophthalmol. 122, 564 (2004).
29. J. C. Barrett, B. Fry, J. Maller, M. J. Daly, Bioimformatics 21, 263 (2005).
S1. R. Klein et al., Ophthalmology 98, 1128 (1991).
S2. M. L. Klein et al., Arch. Ophthalmol. 116, 1082 (1998).
S3. R. Klein, B. E. Klein, S. C. Jensen, S. M. Meuer, Ophthalmology 104, 7 (1997).
S4. J. Majewski et al., Am. J. Hum. Genet. 73, 540 (2003).
S5. R. Klein, B. E. Klein, K. L. Linton, Ophthalmology 99, 933 (1992).
S6. R. Klein, B. E. Klein, S. C. Tomany, S. M. Meuer, G. H. Huang, Ophthalmology 109, 1767 (2002).
S7. K. J. Livak, Genet. Anal. 14, 143 (1999).
S8. J. B. de Kok, E. T. Wiegerinck, B. A. Giesendorf, D. W. Swinkels, Hum. Mutat. 19, 554 (2002).
S9. A. Bansal et al., Proc. Natl. Acad. Sci. USA 99, 16871 (2002).
S10. J. Dausset et al., Genomics 6, 575 (1990).
S11. K. H. Buetow et al., Proc. Natl. Acad. Sci. USA 98, 581 (2001).
S12. K. L. Mohlke et al., Proc. Natl. Acad. Sci. USA 99, 16928 (2002).
S13. M. R. Nelson et al., Genome Res. 14, 1664 (2004).
S14. C. Jurinke, P. Oeth, D. van den Boom, Mol. Biotechnol. 26, 147 (2004).
S15. D. Thomas, Statistical methods in genetic epidemiology (Oxford University Press, New York, 2004), pp. page 52.
S16. J. C. Barrett, B. Fry, J. Maller, M. J. Daly, Bioinformatics 21, 263 (2005).
S17. S. B. Gabriel et al., Science 296, 2225 (2002).
S18. Nature 426, 789 (2003). Page 32
S19. D. W. Schultz et al., Hum. Mol. Genet. 12, 3315 (2003).
S20. G. R. Abecasis et al., Am. J. Hum. Genet. 74, 482 (2004).
S21. M. Hayashi et al., Ophthalmic. Genet. 25, 111 (2004).
S22. S. K. Iyengar et al., Am. J. Hum. Genet. 74, 20 (2004).
S23. G. J. McKay et al., Mol. Vis. 10, 682 (2004).
S24. E. M. Stone et al., N. Engl. J. Med. 351, 346 (2004).
   All references described herein are incorporated by reference in their entirety.

We claim:

1. A method for determining whether or not an individual has an increased risk of susceptibility to age-related macular degeneration (AMD) comprising:
i. obtaining a biological sample from an individual;
ii. analyzing the biological specimen to determine whether a human complement factor H (CFH) has at least one mutation and/or polymorphism in the human CFH gene or gene product as compared to a control group, wherein the presence of a mutation and/or polymorphism indicates that the individual is at increased risk of developing AMD.

2. The method of claim 1, wherein the mutation and/or polymorphism is in a coding region of the CFH gene.

3. The method of claim 2, wherein the mutation and/or polymorphism results in an amino acid change in the expression product.

4. The method of claim 1, wherein the mutation and/or polymorphism is within a region of CFH encoding a short consensus repeat (SCR).

5. The method of claim 4, wherein the SCR is SCR7.

6. The method of claim 5, wherein the mutation and/or polymorphism encodes amino acid 402 of complement factor H (CFH).

7. The method of claim 6, wherein the mutation and/or polymorphism changes the tyrosine at amino acid 402 to a histidine.

8. The method of claim 1, wherein the mutation and/or polymorphism changes the thymine at position 1277 of SEQ ID NO. 1 to a cytosine.

9. The method of claim 1, wherein the individual is Caucasian and at least 55 years of age.

10. A method of screening for an individual at increased risk of developing an eye disease, comprising:
i. obtaining a biological sample from a subject;
ii. analyzing human complement factor H (CFH) to determine if more mutations and/or polymorphisms in the human CFH gene or gene product in the biological sample are present when compared to a control group, wherein the presence of a mutation and/or polymorphism indicates that the individual is at increased risk of developing AMD.

11. The method of claim 10, wherein the eye disease is an on optic nerve disorder.

12. A method of treating a patient who is has an increased risk for developing AMD, comprising:
i. obtaining a biological sample from an individual;
ii. analyzing the biological specimen to determine whether a human complement factor H (CFH) has at least one mutation and/or polymorphism in the human CFH gene or gene product as compared to a control group, wherein the presence of a mutation and/or polymorphism indicates that the individual is at increased risk of developing AMD; and
iii. administering a treatment for AMD to the patient if the presence of the at least one mutation and/or polymorphism is detected.

13. The method of claim 12, wherein the treatment is selected from the group consisting of an angiogenesis inhibitor, a VEGF inhibitor, photodynamic laser therapy, and pegaptanib sodium.

14. The method of claim 13, wherein the VEGF inhibitor is ranibizumab.


SEQUENCE LISTING (14 sequences)

SEQ ID NO: 1 - DNA, 3926 bp, Homo sapiens
aattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctctt

SEQ ID NO: 2 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - DNA, 45 bp, Artificial Sequence
  Description of Artificial Sequence Synthetic probe
ctttatttatttatcattgttatggtccttaggaaaatgttattt

SEQ ID NO: 4 - DNA, 25 bp, Artificial Sequence
  Description of Artificial Sequence Synthetic probe
ggcaggcaacgtctatagatttacc

SEQ ID NO: 5 - DNA, 17 bp, Artificial Sequence
  Description of Artificial Sequence Synthetic probe
tttcttccatgattttg

SEQ ID NO: 6 - DNA, 16 bp, Artificial Sequence
  Description of Artificial Sequence Synthetic probe
ttcttccataattttg

SEQ ID NO: 7 - DNA, 40 bp, Homo sapiens
aaaatggatataatcaaaattatgaagaaagtttgtacag

SEQ ID NO: 8 - DNA, 201 bp, Homo sapiens
ctgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatgg

SEQ ID NO: 9 - DNA, 3926 bp, Homo sapiens
aattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctctt

SEQ ID NO: 10 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 11 - protein, 58 aa, Mus musculus
VRKCVFHYVENGDSAYWEKVYVQGQSLKVQCYNGYSLQNGQDTMTCTENGWSPPPKCI

SEQ ID NO: 12 - protein, 58 aa, Rattus norvegicus
LRQCIFHYVEYGESLYWQRRYIEGQSAKVQCHSGYSLPNGQDTILCTENGWSPPPKCV

SEQ ID NO: 13 - protein, 58 aa, Homo sapiens
LRKCYFPYLENGYNQNYGRKFVQGKSIDVACHPGYALPKAQTTVTCMENGWSPTPRCI

SEQ ID NO: 14 - protein, 54 aa, Gallus gallus
LKRCDVGPENGYIQNNWRYFKEGERTYFCNKNYRTNEDGEITCSNGWSPTPRCI